(19) United States
(12) Patent Application Publication
(10) Pub. No.: US 2023/0332191 A1
(43) Pub. Date: Oct. 19, 2023

(54) SYNTHETIC GROWTH ON ONE-CARBON SUBSTRATES

(71) Applicant: UNIVERSITY OF SOUTH FLORIDA, Tampa, FL (US)

(72) Inventors: Ramon GONZALEZ, Tampa, FL (US); Alexander CHOU, Tampa, FL (US); James CLOMBURG, Tampa, FL (US); Fayin ZHU, Tampa, FL (US); Seung Hwan LEE, Tampa, FL (US)

(21) Appl. No.: 18/042,589

(22) PCT Filed: Aug. 26, 2021
(86) PCT No.: PCT/US2021/047765

§ 371 Date: Feb. 22, 2023

Related U.S. Application Data
(60) Provisional application No. 63/070,464, filed on Aug. 26, 2020.

(51) Int. Cl. C12P 7/56; C12P 7/42; C12N 15/52; C12N 9/04; C12N 9/02; C12N 9/10; C12N 9/12

(52) U.S. Cl. CPC C12P 7/56; C12P 7/42; C12N 15/52; C12N 9/0006; C12Y 101/01244; C12N 9/0008; C12Y 102/01; C12Y 102/05; C12Y 102/01046; C12N 9/0071; C12Y 114/18003; C12N 9/0093; C12Y 117/01; C12N 9/1029; C12Y 203/01; C12N 9/1217; C12Y 207/02006

(57) ABSTRACT

Many biotechnologically relevant organisms cannot utilize cheap and abundant one carbon feedstocks, e.g. CO₂, CO, formaldehyde, methanol, and methane, for growth and instead prefer complex feedstocks such as sugars. Disclosed herein is a system that enables organisms to consume one carbon molecules for growth and maintenance via a formyl-CoA elongation pathway. Utilization of one carbon feedstocks can replace the use of sugar as the primary means of cultivating organisms in biotechnological applications. This has the potential to be more cost effective and avoid the controversial use of food as feedstocks. Intermediates of the formyl-CoA elongation pathway may be also be converted to desired chemical products.

### CROSS-REFERENCE TO RELATED APPLICATIONS

This application is related to, claims priority to, and incorporates herein by reference for all purposes U.S. Provisional Patent Application No. 63/070,464, filed on Aug. 26, 2020.

### BACKGROUND

One-carbon (C1) compounds represent potential low-cost and abundant feedstocks for the chemical industry (Dürre, P. & Eikmanns, B J. Curr. Opin. Biotechnol. 35:63-72 (2015)). Due to the often dilute and disperse nature of these feedstocks, biochemical processes have the potential to be effective technologies for C1 utilization by enabling lower capital expenditure (CapEx) and distributed manufacturing in ways that current chemical technologies are limited (Clomburg, J M., et al. Science 355:aag0804 (2017)). While C1 molecules can be effectively utilized by biology for growth, the efficient biological production of varied industrial chemicals from C1 substrates remains an open challenge.

Approaches toward biological product synthesis from C1 substrates involving both natural (Bar-Even, A., et al. J. Exp. Bot. 63:2325-42 (2012); Kalyuzhnaya, M G., et al. Metab. Eng. 29:142-152 (2015)) and synthetic (Bogorad, I. W. et al. Proc. Natl. Acad. Sci. U.S.A 111:15928-33 (2014); Siegel, J. B. et al. Proc. Natl. Acad. Sci. 112: 3704-3709 (2015); Schwander, T., et al. Science 354:900-904 (2016); Lu, X. et al. Nat. Commun. 10: 1378 (2019); Kim, S. et al. Nat. Chem. Biol. 16:538-545 (2020)) pathways tend to share a common metabolic architecture (FIG. 1A). C1 molecules at varying levels of reduction are first assimilated to produce C2 and C3 central metabolites. These C2/C3 metabolites are either precursors for product synthesis pathways or are further converted through central metabolic pathways to generate said precursors. As a result, carbon assimilation, central metabolism, and product synthesis pathways must be concurrently engineered to enable effective production of targeted chemicals. Conceptually, though, this architecture should not be a necessity for chemical production from C1 molecules. Unlike the utilization of multi-carbon substrates for which there is an advantage to conserve carbon-carbon bonds, all carbon-carbon bonds must be created de novo when starting from C1 molecules. In principle, then, it should be possible to build diverse chemical products from C1 molecules using C1 “building blocks” without the added complexity of first generating multi-carbon units.

While fuel and chemical production based on C1 feedstocks is promising, significant challenges impede the implementation of efficient biocatalysts for C1 bioconversion. Many biotechnologically relevant organisms cannot utilize cheap and abundant one carbon feedstocks (e.g. CO₂, methane) for growth and instead prefer complex feedstocks such as sugar.

### SUMMARY

As disclosed herein, formyl-CoA can serve as a C1 building block or elongation unit in a reaction catalyzed by 2-hydroxyacyl-CoA lyase (HACL) or oxalyl-CoA decarboxylase (OXC) allowing organisms to utilize C1 feedstocks for growth and resulting in a more cost-effective way of cultivating said organisms. These pathways, referred to herein as formyl-CoA elongation (FORCE) pathways, can be used for the production of growth substrates for biocatalyst growth or maintenance and ultimately for biochemical product synthesis. In some embodiments, the disclosed FORCE pathways can be used with multi-carbon substrates, such as C2, C3, C4, C5, C6 substrates. For example, multicarbon co-substrates can include for example: sugars (e.g. glucose), glycerol, acetate, and fatty acids.

Therefore, disclosed herein are microorganisms that are not naturally able to utilize C1 substrates for growth (i.e. heterotrophs) but which have been engineered to be able to do so. Engineering of these organisms, which are referred to as either methylotrophs, formatotrophs, or autotrophs, involves providing a cell system a first set of metabolic enzymes to convert the single carbon substrate to formyl-CoA and formaldehyde, a second set of metabolic enzymes to elongate aldoses or aldehydes with the formyl-CoA molecules, feeding the system a C1 substrate under suitable conditions for the metabolic enzymes to produce multi-carbon native substrates or metabolites, and optionally providing a third set of metabolic enzymes to convert substrates or metabolites into a desired multi-carbon chemical.

Disclosed herein is a non-natural microbial system capable of utilizing one-carbon (C1) substrates for growth and product synthesis. The non-natural microbial system may include a first set of nucleic acids encoding enzymes to convert the single carbon substrate to formyl-CoA and formaldehyde, and a second set of nucleic acids encoding enzymes to convert formyl-CoA and formaldehyde to native multi-carbon substrates or metabolites that enable growth.

Further disclosed herein is a metabolically engineered microorganism. The metabolically engineered microorganism may include a first set of nucleic acids encoding metabolic enzymes that convert a single carbon substrate to formyl-CoA, and a second set of nucleic acids encoding metabolic enzymes that extend a carbon backbone via a formyl-CoA elongation pathway that uses the formyl-CoA as an elongation unit.

Also disclosed herein is a method of cultivating a microorganism on a single carbon substrate. The method may include providing the microorganism with a first set of nucleic acids encoding metabolic enzymes for converting the single carbon substrate to formyl-CoA, and a second set of nucleic acids encoding metabolic enzymes for extending a carbon backbone via a formyl-CoA elongation pathway that uses the formyl-CoA as an elongation unit. The method may further include culturing the microorganism in a growth medium containing the single carbon substrate. One or more intermediates of the formyl-CoA elongation pathway may serve as a growth substrate or a precursor to a growth substrate of the microorganism.

Further disclosed herein is a method of chemical product synthesis from a single carbon substrate. The method may include providing a microorganism with a first set of nucleic acids encoding metabolic enzymes that convert the single carbon substrate to formyl-CoA, and a second set of nucleic acids encoding metabolic enzymes that extend a carbon backbone via a formyl-CoA elongation pathway that uses the formyl-CoA as an elongation unit. The method may further include feeding the microorganism the single carbon substrate. One or more intermediates of the formyl-CoA elongation pathway may be a chemical product or may serve as a precursor to a chemical product.

Also disclosed herein is a cell-free system including a first set of metabolic enzymes that convert a single carbon substrate to formyl-CoA, and a second set of metabolic enzymes that extend a carbon backbone via a formyl-CoA elongation pathway that uses the formyl-CoA as an elongation unit.

Also disclosed herein is a two-strain microbial system. The two-strain microbial system may include a first microorganism including nucleic acids encoding one or more first metabolic enzymes that convert a single carbon substrate to formyl-CoA, and nucleic acids encoding one or more second metabolic enzymes that produce glycolate from the formyl-CoA. The first microorganism may be unable to consume and grow on the glycolate. The two-strain microbial system may further include a second microorganism lacking nucleic acids encoding the first and second metabolic enzymes. The second microorganism may be able to consume and grow on glycolate. Coculturing the first microorganism and the second microorganism in media containing the single carbon substrate may lead to growth of the second microorganism.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. FORCE pathways for product synthesis from C1 substrates. a) A synthetic, orthogonal architecture for C1 utilization based on formyl-CoA elongation (FORCE) pathways. Carbon skeletons are directly built from activated C1 units in the form of formyl-CoA, thus bypassing the “bowtie” architecture of metabolism for product synthesis. b) One-carbon substrates are activated to the C1 elongation unit formyl-CoA through various redox reactions (blue box). Formyl-CoA serves to elongate an aldehyde in a reaction catalyzed by HACL, resulting in the production of 2-hydroxyacyl-CoA. 2-Hydroxyacyl-CoA can be further reduced to a 2-hydroxyaldehyde. The 2-hydroxyaldehyde can be further elongated by formyl-CoA, which we refer to as aldose elongation. Alternatively, α-reduction can take place via reduction to a 1.2-diol and dehydration to a nonfunctionalized aldehyde. The resulting aldehyde can then be further elongated. These collective routes for elongation, referred to as formyl-CoA elongation (FORCE), are boxed in green. The various intermediates of these elongation pathways can be converted to desirable chemical products (red) including 2-hydroxy-acids, aldoses, diols, polyols, carboxylic acids, and alcohols. A number of these products and intermediates can also serve as substrates for growth (highlighted in orange), such as glycolic acid, glyceraldehyde, and acetyl-CoA. Abbreviations: MDH: methanol dehydrogenase; ACR: acyl-CoA reductase; FaldDH: formaldehyde dehydrogenase; ACS: acyl-CoA synthetase; ACT: acyl-CoA transferase; FOK: formate kinase; PTA: phosphotransacylase; HACL: 2-hydroxyacyl-CoA lyase; ADH: alcohol dehydrogenase; DDR: diol dehydratase; TES: thioesterase; ALDH: aldehyde dehydrogenase. Standard Gibbs free energies of reactions are given for each pathway reaction in the direction indicated by the arrow.

FIG. 2. Thermodynamic analysis of FORCE pathways. Thermodynamic feasibility was evaluated by calculating the Min-max Driving Force (MDF) of specified conversions. a) The MDF for the utilization of different C1 substrates for the production of glycolate or acetate via the synthetic pathway. Open bars refer to the standard conditions (maximum substrate concentration constraint 10 mM), while filled bars refer to adjusted constraints reflecting the approximate toxicity of each substrate to E. coli. Three pathways for formate utilization were evaluated, each different in the ATP requirement for formate activation. b) The influence of factors such as NADH/NAD⁺ ratio, ATP consumption, and formate concentration on the MDF of formate to product conversion. c) The MDF of iterative FORCE pathways as a function of the product carbon chain length using formaldehyde as the representative substrate and where the product is the aldose or aldehyde corresponding to the indicated chain length. These aldose and aldehyde elongation pathways are shown in FIG. 2 (see formyl-CoA elongation panel).

FIG. 3. In vitro assessment of core module of the FORCE pathway using purified enzymes. a) Pathways for conversion of C1 substrates formaldehyde and formate (individually and in combination) to glycolyl-CoA. Enzymes and co-factors for each step are indicated. Substrate(s) added are shown in bold and underlined. b) A Liquid Chromatography-Mass Spectrometry (LC-MS) extracted ion chromatography (EIC) of formyl-CoA and glycolyl-CoA through Find By Formula (FBF) function in MassHunter Qualitative Analysis B.05.00. The data is representative of duplicate experiments; c) Relative abundance of formyl-CoA and glycolyl-CoA in the in vitro samples. Formyl-CoA and glycolyl-CoA were quantified based on EIC peak area by LC-MS analysis. Resulting C2 in the samples were further quantified as glycolic acid after NaOH hydrolysis of glycolyl-CoA. All data is shown for technical replicates (n=2) with bars drawn to the mean values.

FIG. 4. Cell-free prototyping the α-reduction variant of the FORCE product synthesis pathway. a) Overview of the prototyped α-reduction pathway for the production of various C2 products from formaldehyde. Products that were detected in this work are boxed with a solid outline. Enzyme abbreviations: DDR: Klebsiella oxytoca diol dehydratase; End. (1): endogenous aldehyde dehydrogenase; End. (2): endogenous thioesterase; End. (3): endogenous alcohol dehydrogenase; FucO: E. coli 1.2-diol oxidoreductase; LmACR: Listeria monocytogenes acyl-CoA reductase; RuHACL: Rhodospirillales bacterium URHD0017 HACL. b) Product and substrate profiles of cell-free systems with indicated pathway enzymes as detected by HPLC under conditions in which carboxylates are detected in their acid form. Concentrations are given on a carbon basis. All data points are shown for triplicate technical replicates. Lines are drawn to the mean values with error bars indicating the standard deviation.

FIG. 5. Resting cell bioconversions of C1 substrate formaldehyde using the aldose elongation and α-reduction variants of the FORCE pathways. a) Strategies used in this work to demonstrate diverse product synthesis using FORCE pathways from formaldehyde. Detected products and byproducts are boxed with a solid outline. Knockout strategies to reduce byproduct synthesis are indicated in red. b) Metabolite profiles for strains engineered for product synthesis from formaldehyde using FORCE pathways after 24 hour resting cell bioconversions with OD₆₀₀=10 (5*10⁹ CFU/mL) and two additions of 10 mM formaldehyde at 0 and 1.5 hours. In the legend, +refers to overexpression of the indicated enzyme. Δaldh refers to knockouts of aldehyde dehydrogenases: ΔaldA ΔaldB ΔpatD ΔpuuC. End. tes refers to endogenous thioesterases and spontaneous thioester hydrolysis. No multi-carbon products were observed in a strain that was expressing LmACR and EcAldA only without RuHACL. Concentrations are given on a carbon basis and were determined by HPLC under conditions in which carboxylates are detected in their acid form. All data points are shown for duplicate technical replicates. Bars are drawn to the mean values. c) Spectra of multi-carbon products generated from experiments using 13C-labeled formaldehyde in comparison to products from unlabeled formaldehyde. The [M−15]⁺ ion is shown. A +2 shift in m/z is observed for glycolic acid and ethylene glycol, and a +3 shift in m/z is observed for glyceric acid.

FIG. 6. FORCE pathway implementation in growing cell cultures using methanol as the C1 substrate. a) Host and pathway designs for the production of glycolate from methanol in actively growing E. coli cultures. Knockout strategies to reduce byproduct synthesis and prevent glycolate utilization are indicated in red and correspond to host strain AC440. ATE refers to knockouts of endogenous thioesterases (ΔyciA ΔtesA ΔtesB ΔybgC Δydil ΔfadM). End. (1) refers to endogenous aldehyde oxidation activity. Enzyme abbreviations: BmMDH2^{MGA3}: Bacillus methanolicus MGA3 NAD⁺-dependent methanol dehydrogenase; LmACR: Listeria monocytogenes acyl-CoA reductase; RuHACL^{G390N}: Rhodospirillales bacterium URHD0017 HACL (G390N); BsmHACL: Beach sand metagenome HACL; EcAldA: E. coli aldehyde dehydrogenase A; CbAbfT: Clostridium aminobutyricum CoA transferase. b) Time course of production of glycolate and formate from methanol. FORCE pathway designs were implemented by overexpressing LmACR, EcAldA, and BmMdh^{MGA3} with or without RuHACL^{G390N}. All data is shown for biological replicates (n=3 for samples with RuHACL^{G390N}; n=2 for samples without RuHACL^{G390N}). Lines are drawn to the mean values with error bars indicating the standard deviation. Concentrations are given on a carbon basis. c) Improvement of glycolate production from methanol in growing E. coli cultures via rational engineering. Glycolate and formate concentrations are given on a carbon basis for the 72-hour time point. All data is shown for biological replicates (n=3 for samples with RuHACL^{G390N}; n=4 for others). Bars are drawn to the mean values. d) Spectra of the [M−15]⁺ ion of the 2TMS derivative of glycolic acid produced by E. coli incubated with either 12C (unlabeled) or 13C (labeled) methanol.

FIG. 7. Simulated flux maps from genome scale E. coli models for growth using FORCE pathways variants: a) (form)aldehyde elongation, b) α-reduction, c) aldose elongation. Substrate uptake reactions are indicated in green. The reactions implemented for each FORCE pathway variant are drawn in blue. FORCE pathway termination is indicated in orange. Carbon dissimilated as CO₂ export is highlighted in red. Fluxes are given in mmol/g DCW/hr. Only major fluxes (threshold set as >μ) are drawn for clarity. Reactions of the pentose phosphate pathway, resulting in the rearrangement of erythrose 4-phosphate into glyceraldehyde 3-phosphate in panel b are simplified.

FIG. 8. Two-strain system for evaluating the ability of FORCE pathways to enable growth on C1 substrates. a) FORCE pathways can enable synthetic methylotrophy by converting non-native C1 substrates into native multi-carbon substrates that serve as carbon and energy sources. b) Conceptual scheme of the two-strain system. Producer strains (yellow outline) that are unable to consume glycolate were engineered to produce glycolate from one of three C1 substrates: methanol (red), (para)formaldehyde (blue), or formate and formaldehyde (green). A second consumer strain capable of consuming glycolate was added to the culture, acting as a detectable signal to evaluate growth. c) Time course of glycolate concentration (blue) and cell-growth (orange) in the two-strain system with (para)formaldehyde as the sole source of carbon. 5 mM (mass equivalent) paraformaldehyde added to AC440 (3*10⁹ CFU/mL) expressing LmACR, AIdA, and BsmHACL. All data points are shown for duplicate replicates. The line for glycolate concentration is drawn to the mean values. The line for cell growth is the fit of the data to exponential growth by least squares regression, which was used to calculate the specific growth rate (μ). Full metabolite and cell growth profiles, including for control samples are shown in FIG. 16. d) Growth of the consumer strain when incubated for the indicated time with the relevant producer strain with (+) or without (−) HACL and the indicated C1 substrate (pFALD: paraformaldehyde; MeOH: methanol; FALD: formaldehyde; FA: sodium formate). See also FIG. 16-18. All data is shown for duplicate technical replicates with bars drawn to the mean values. e) Plate images demonstrating growth of the consumer strain corresponding to the conditions in panel d.

FIG. 9. Canonical (a) and orthogonal, synthetic (b) architectures for biological C1 utilization. a) “Bowtie” architecture of metabolism in which carbon substrates are consolidated into central metabolites from which a host of products can be produced through fermentative and biosynthetic pathways. Metabolic engineering typically operates within this framework by manipulating either one or all of the three components of the bowtie. b) The orthogonal FORCE pathways serve as a platform for both product synthesis and for providing substrates/metabolites for growth. This is an alternative framework to the traditional approach, which feeds all carbon through central metabolism, and from which both products and biomass are derived.

FIG. 10. An alternative FORCE pathway based on dehydration of the 2-hydroxyacyl-CoA and α-reduction. The pathway resembles β-oxidation reversal (β-reduction)³⁹. This pathway is also a potential route for the production of unsaturated products. HACL: 2-hydroxyacyl-CoA lyase; HACD: 2-hydroxyacyl-CoA dehydratase; TER: trans-2-enoyl-CoA reductase; ACR: acyl-CoA reductase.

FIG. 11. The impact of NADH/NAD⁺ ratio on formaldehyde (top) and methanol (bottom) conversion to glycolate or acetate via FORCE pathways.

FIG. 12. The impact of NADH/NAD⁺ ratio on formaldehyde (top) and methanol (bottom) conversion to glycolate or acetate via FORCE pathways. Termination by hydrolysis of the acyl-CoA to produce sugar acids increases the driving force of the pathway for low numbers of iterations, but the driving forces converge as the number of iterations increase.

FIG. 13. Production of glycolate from formate by E. coli engineered with a formate-activating pathway. Resting cell experiments were performed with a strain expressing CaAbfT and BsmHACL (blue bars) and the corresponding control lacking BsmHACL (orange bars). Cultures (2.5 OD₆₀₀=2.5*10⁹ CFU/mL) were incubated at 30° C. for 24 hours in 25 mL flasks shaking at 200 rpm using 10 mM formate (plus 1 mM formaldehyde) as carbon source (control cultures with 1 mM formaldehyde and no formate also shown). All data points are shown for n=6 replicates. Bars are drawn to the mean values.

FIG. 14. Predicted biomass electron and carbon yields from various C1 substrates by the implementation of select pathways enabling methylotrophy. Abbreviations: Formald—formaldehyde, FORCE-Glycerald—FORCE pathway with reactions enabling glyceraldehyde production, RuMP—Ribulose monophosphate pathway, FORCE-Ac—FORCE pathway with reactions enabling acetate production, SACA—Synthetic Acetyl-CoA pathway, FORCE-Glycolate—FORCE pathway with reactions enabling glycolate production. The scenarios in bold correspond to the predicted flux maps illustrated in FIG. 8.

FIG. 15. Paraformaldehyde solubilization rate and resting cell bioconversion with paraformaldehyde. a) Solubilization rate of commercially available paraformaldehyde (pFALD) with different particle sizes. Solubilization rates are measured in 10 mL M9 media in a 25 mL flask at 30° C. shaking at 200 rpm. b) Resting cell bioconversion of strains expressing BsmHACL, LmACR and AldA induced with 40 μM cumate and 100 μM IPTG. 3 mg prilled paraformaldehyde is added to 20 mL M9 media (2.5 mM formaldehyde equivalent) in a 25 mL flask at 30° C. shaking at 200 rpm. Formaldehyde accumulates only at sub-millimolar concentrations under these conditions.

FIG. 16. Time course profiles for glycolate, formate, and formaldehyde concentration and cell-growth of the sensor strain in the two-strain system with 5 mM paraformaldehyde. a) Time course in which the producer strain did not express an HACL. b) Plates from a representative experiment of the time course shown in panel a. c) Time course in which the producer strain expresses HACL. d) Plates from a representative experiment of the time course shown in panel c. 50 μL of cultures (5×10⁻³ dilution) at various time points plated on minimal media plates containing 2.5 g/L glycolate. All data is shown for duplicate replicates (n=2). Lines are drawn to the mean values.

FIG. 17. Time course profiles for glycolate, formate, and formaldehyde concentration and cell-growth of the sensor strain in the two-strain system with 500 mM methanol. a) Time course in which the producer strain did not express an HACL. b) Plates from a representative experiment of the time course shown in panel a. c) Time course in which the producer strain expresses HACL. d) Plates from a representative experiment of the time course shown in panel c. 50 μL of cultures (5×10⁻³ dilution) at various time points plated on minimal media plates containing 2.5 g/L glycolate. All data is shown for duplicate replicates (n=2). Lines are drawn to the mean values.

FIG. 18. Time course profiles for glycolate, and formaldehyde concentration in the two-strain system with 1 mM formaldehyde and 10 mM formate. a) Time course in which the producer strain expresses BsmHACL. b) Plates from a representative experiment of the time course shown in panel a. 50 μL of cultures (5×10⁻³ dilution) at various time points plated on minimal media plates containing 2.5 g/L glycolate. All data is shown for duplicate replicates (n=2). Lines are drawn to the mean values.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As defined herein, the phrases “recombinant host microorganism”, “genetically engineered host microorganism”, “engineered host microorganism” and “genetically modified host microorganism” may be used interchangeably and refer to host microorganisms that have been genetically modified to (a) express one or more exogenous polynucleotides, (b) over-express one or more endogenous and/or one or more exogenous polynucleotides, such as those included in a vector, or which have an alteration in expression of an endogenous gene or (c) knock-out or down-regulate an endogenous gene. In addition, certain genes may be physically removed from the genome (e.g., knock-outs) or they may be engineered to have reduced, altered or enhanced activity.

The terms “engineer”, “genetically engineer” or “genetically modify” refer to any manipulation of a microorganism that results in a detectable change in the microorganism, wherein the manipulation includes, but is not limited to, introducing non-native metabolic functionality via heterologous (exogenous) polynucleotides or removing native-functionality via polynucleotide deletions, mutations or knock-outs. The term “metabolically engineered” generally involves rational pathway design and assembly of biosynthetic genes (ORFs), genes associated with operons, and control elements of such polynucleotides, for the production of a desired metabolite. “Metabolically engineered” may further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway.

The phrases “metabolically engineered microorganism” and “modified microorganism” are used interchangeably herein, and refer not only to the particular subject host cell, but to the progeny or potential progeny of such a cell. Because certain modifications may occur in succeeding generations due to either mutation or environmental influences, such progeny may not, in fact, be identical to the parent cell, but are still included within the scope of the term as used herein.

The term “mutation” as used herein indicates any modification of a nucleic acid and/or polypeptide which results in an altered nucleic acid or polypeptide (i.e., relative to the wild-type nucleic acid or polypeptide sequence). Mutations include, for example, point mutations, substitutions, deletions, or insertions of single or multiple residues in a polynucleotide (or the encoded polypeptide), which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In certain embodiments, a portion of a genetically modified microorganism's genome may be replaced with one or more heterologous (exogenous) polynucleotides. In some embodiments, the mutations are naturally-occurring. In other embodiments, the mutations are the results of artificial selection pressure. In still other embodiments, the mutations in the microorganism genome are the result of genetic engineering.

The term “expression” or “expressed” with respect to a gene sequence, an ORF sequence or polynucleotide sequence, refers to transcription of the gene, ORF or polynucleotide and, as appropriate, translation of the resulting mRNA transcript to a protein. Thus, as will be clear from the context, expression of a protein results from transcription and translation of the open reading frame sequence. The level of expression of a desired product in a host microorganism may be determined on the basis of either the amount of corresponding mRNA that is present in the host, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press, 1989). Protein encoded by a selected sequence can be quantitated by various methods (e.g., by ELISA, by assaying for the biological activity of the protein, or by employing assays that are independent of such activity, such as western blotting or radioimmunoassay, using antibodies that are recognize and bind reacting the protein).

The term “endogenous”, as used herein with reference to polynucleotides (and the polypeptides encoded therein), indicates polynucleotides and polypeptides that are expressed in the organism in which they originated (i.e., they are innate to the organism). In contrast, the terms “heterologous” and “exogenous” are used interchangeably, and as defined herein with reference to polynucleotides (and the polypeptides encoded therein), indicates polynucleotides and polypeptides that are expressed in an organism other than the organism from which they (i.e., the polynucleotide or polypeptide sequences) originated or where derived.

The term “feedstock” is defined as a raw material or mixture of raw materials supplied to a microorganism, or fermentation process, from which other products can be made. For example, as set forth in the present invention, a methane carbon source or a methanol carbon source or a formaldehyde carbon source, either alone or in combination, are feedstocks for a microorganism that produces a bio-fuel or bio-based chemical in a fermentation process. However, in addition to a feedstock (e.g., a methane substrate) of the invention, the fermentation media contains suitable minerals, salts, cofactors, buffers and other components, known to those skilled in the art, suitable for the growth of the cultures and promotion of the enzymatic pathways necessary for multi-carbon compound production.

The term “substrate” refers to any substance or compound that is converted, or meant to be converted, into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material (e.g., methane), but also intermediate and end product metabolites used in a pathway associated with a metabolically engineered microorganism as described herein.

The term “native multi-carbon substrate” as used herein refers to multi-carbon compounds that serve as a growth substrate or metabolite that enables growth of a microorganism.

The term “fermentation” or “fermentation process” is defined as a process in which a host microorganism is cultivated in a culture medium containing raw materials, such as feedstock and nutrients, wherein the microorganism converts raw materials, such as a feedstock, into products.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) of more codons representing amino acids, and (iii) a termination codon, the ORF being read (or translated) in the 5′ to 3′ direction.

It is understood that the polynucleotides described herein include “genes” and that the nucleic acid molecules described herein include “vectors” or “plasmids”. Accordingly, the term “gene”, refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “promoter” refers to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “codon-optimized” as it refers to genes or coding regions of nucleic acid molecules (or ORFs) for transformation of various hosts, refers to the alteration of codons in the gene or coding regions of the nucleic acid molecules to reflect the typical codon usage of the host organism without altering the polypeptide encoded by the DNA.

The term “operon” refers to two or more genes which are transcribed as a single transcriptional unit from a common promoter. In certain embodiments, the genes, polynucleotides or ORFs comprising the operon are contiguous genes. It is understood that transcription of an entire operon can be modified (i.e., increased, decreased, or eliminated) by modifying the common promoter. Alternatively, any gene, polynucleotide or ORF, or any combination thereof in an operon can be modified to alter the function or activity of the encoded polypeptide. The modification can result in an increase or a decrease in the activity or function of the encoded polypeptide. Further, the modification can impart new activities on the encoded polypeptide.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes”, that is, that replicate autonomously or can integrate into a chromosome of a host microorganism. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The term “homolog”, as used with respect to an original enzyme, polypeptide, gene or polynucleotide (or ORF encoding the same) of a first family or species, refers to distinct enzymes, genes or polynucleotides of a second family or species, which are determined by functional, structural or genomic analyses to be an enzyme, gene or polynucleotide of the second family or species, which corresponds to the original enzyme or gene of the first family or species. Most often, “homologs” will have functional, structural or genomic similarities. Techniques are known by which homologs of an enzyme, gene or polynucleotide can readily be cloned using genetic probes and PCR. Identity of cloned sequences as “homologs” can be confirmed using functional assays and/or by genomic mapping of the genes.

A polypeptide (or protein or enzyme) has “homology” or is “homologous” to a second polypeptide if the nucleic acid sequence that encodes the polypeptide has a similar sequence to the nucleic acid sequence that encodes the second polypeptide. Alternatively, a polypeptide has homology to a second polypeptide if the two proteins have “similar” amino acid sequences. Thus, the terms “homologous proteins” or “homologous polypeptides” is defined to mean that the two polypeptides have similar amino acid sequences. In certain embodiments of the invention, polynucleotides and polypeptides homologous to one or more polynucleotides and/or polypeptides set forth in Table 1 may be readily identified using methods known in the art for sequence analysis and comparison.

The term “CoA” as used herein refers to coenzyme A.

A homologous polynucleotide or polypeptide sequence of the invention may also be determined or identified by BLAST analysis (Basic Local Alignment Search Tool) or similar bioinformatic tools, which compare a query nucleotide or polypeptide sequence to a database of known sequences. For example, a search analysis may be done using BLAST to determine sequence identity or similarity to previously published sequences, and if the sequence has not yet been published, can give relevant insight into the function of the DNA or protein sequence.

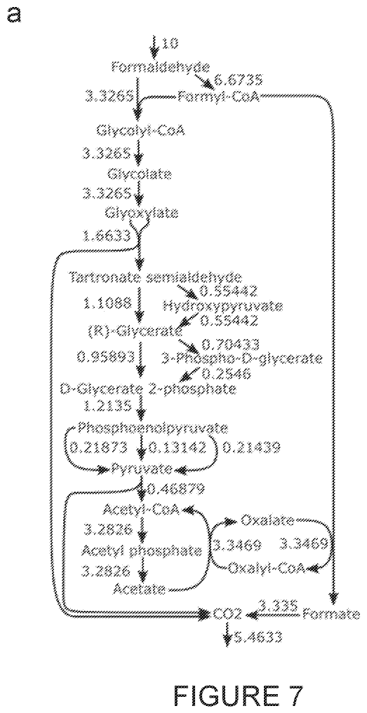

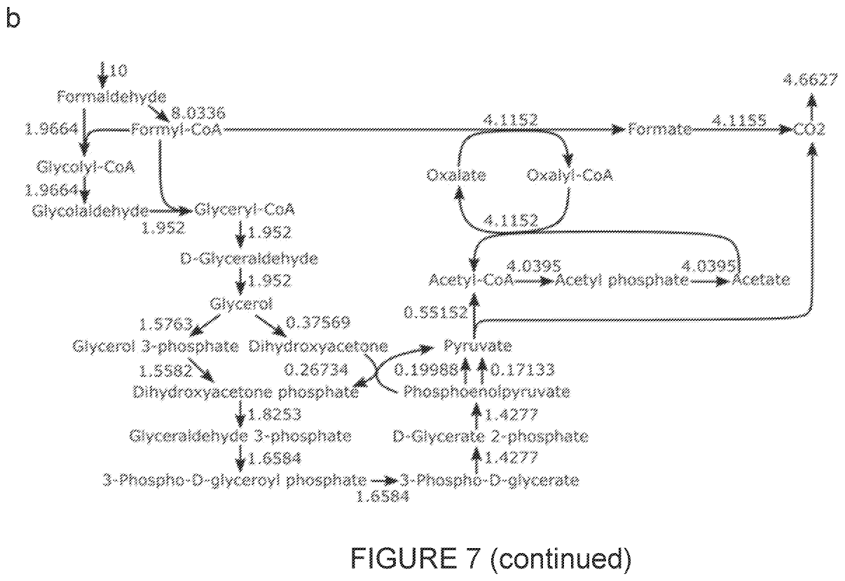

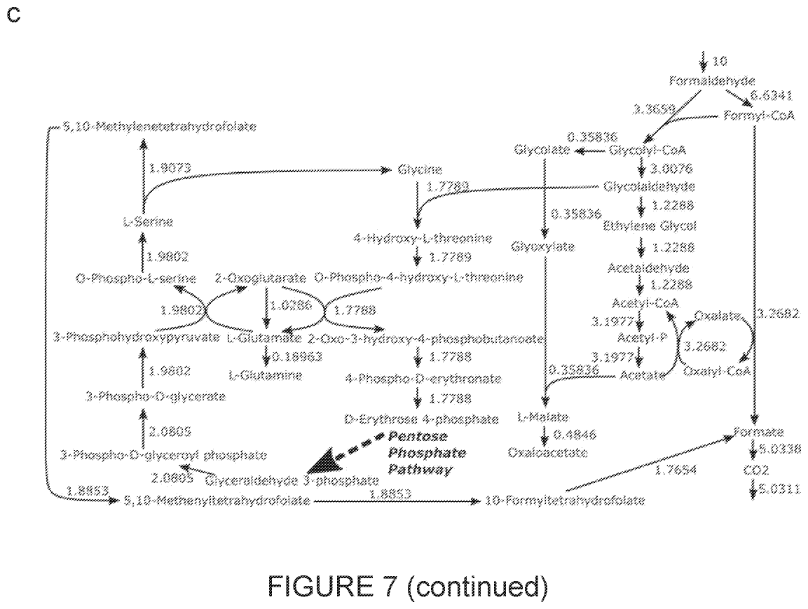

The current invention provides systems and microorganisms engineered to endow them with pathways that enable growth on C1 substrates (without said engineered/synthetic pathways, said microorganisms are not able to grow on any C1 substrate). In some embodiments, the system comprises a C1 substrate and modified organisms capable of growth on C1 substrates. This invention provides systems, organisms, and methods of conversion of C1 substrates to cells (i.e. growth on C1 substrates). As demonstrated in the Examples and FIGS. 7-9, 14, and 16-18 (and material that relates to these figures) demonstrate growth on C1 substrates.

In various embodiments, the invention provides for the single carbon (C1) compound serving as a source of both energy and carbon for the organism. Single carbon molecules of various reduction levels are interconverted to produce formyl-CoA, the single carbon unit used to extend a carbon backbone. Systems and methods for bioconversion of C1 feedstocks based on the use of formate acyltransferases are described in WO 2017/210381, which is incorporated by reference for these teachings. In contrast, the disclosed system uses formyl-CoA as the C1 building block or elongation unit in a reaction catalyzed by 2-hydroxyacyl-CoA lyase (HACL). This approach is both simpler in design (fewer overall reaction steps) and in practice (increased oxygen tolerance).

In some embodiments, single carbon (C1) molecules are the solely supplied carbon source. In these situations, a one-carbon acyl-CoA, formyl-CoA, is produced. In some embodiments, formate can be converted to formyl-CoA either directly by a suitable acetyl-CoA synthetase or through the intermediate formyl-phosphate by a suitable formate kinase and phosphate acetyl-transferase. Formaldehyde can also be converted to formyl-CoA by a suitable acyl-CoA reductase.

Combinations of the above reactions can be used to generate formyl-CoA from other single carbon molecules. For example, an implementation that makes use of methane would include the expression of a methane monooxygenase, a methanol dehydrogenase, and an acyl-CoA reductase. Even more combinations of the described reactions and accompanying enzymes can be used to allow for implementations that use a mixture of single carbon units, for example a combination of methane and carbon dioxide through all of the described reactions. At a minimum, this function can be accomplished from either formaldehyde, by the expression of an acylating aldehyde dehydrogenase, or from formate, by a suitable acetyl-CoA synthetase or combined formate kinase and phosphate acetyl-transferase.

Therefore, disclosed herein is a method for enabling a heterotroph to utilize single carbon (C1) substrates (e.g. methane, methanol, carbon dioxide, formate, formaldehyde) for growth comprising the steps of providing a cell system containing a first set of metabolic enzymes to convert the single carbon substrate to formyl-CoA and formaldehyde, a second set of metabolic enzymes to elongate aldoses or aldehydes (including the produced formaldehyde) with the formyl-CoA molecules, feeding the system a C1 substrate under suitable conditions for the metabolic enzymes to produce aldehyde or aldose intermediates of desired carbon lengths, and optionally providing a third set of metabolic enzymes to convert the aldehyde or aldose intermediates into the desired multi-carbon chemical.

The first step in the disclose systems and methods is the conversion of the single carbon substrate (e.g. methane, methanol, carbon dioxide, formate, formaldehyde) into formyl-CoA and formaldehyde. This step is referred to herein as C1 activation.

In general, the conversion of methane (CH₄) to formaldehyde (H₂C═O) and formyl-CoA requires at least the following three steps: (1) the methane (CH₄) substrate is first oxidized to methanol (CH₃OH) via a methane monooxygenase (MMO; EC 1.14.13.25), (2) the methanol (CH₃OH) is then oxidized to formaldehyde (H₂C═O) via a methanol dehydrogenase (MDH; E.C. 1.1.1.244, 1.1.2.7), and (3) some of the formaldehyde (H₂C═O) is oxidized to formyl-CoA via an acyl-CoA reductase (ACR). Exemplary Acyl-CoA reductases or acyling aldehyde dehydrogenases include fatty acyl-CoA reductase (EC 1.2.1.84), succinyl-CoA reductase (EC 1.2.1.76), acetyl-CoA reductase, butyryl-CoA reductase, propionyl-CoA reductase (EC 1.2.1.10).

In general, the conversion of carbon dioxide (CO₂) to formyl-CoA first requires that the CO₂ substrate be reduced to formate (HCOO—) via formate dehydrogenase (E.C. 1.2.1.2). The produced formate can then be converted to formyl-CoA by one of three pathways. In some embodiments, the formate is converted to formyl-CoA via acyl-CoA synthetase (ACS; E.C. 6.2.1.1). In some embodiments, the formate is converted to formaldehyde (H2C═O) by formaldehyde dehydrogenase (FaIdDH; E.C. 1.2.1.46), which is then oxidized to formyl-CoA via acyl-CoA reductase (ACR; E.C. E.C. 1.2.1.-, e.g. 1.2.1.10, 1.2.1.76, 1.2.1.84). In some embodiments, the formate is converted to formyl-phosphate via formate kinase (FOK; E.C. 2.7.2.6), which is then converted to formyl-CoA via phosphotransacylase (PTA; EC 2.3.1.8).

As disclosed herein, formyl-CoA can be used as the C1 building block or elongation unit in a reaction catalyzed by a 2-hydroxyacyl-CoA lyase (HACL) or an oxalyl-CoA decarboxylase (OXC; E.C. 4.1.1.8). These enzymes can ligate formyl-CoA with a variety of carbonyl-containing acceptors of broad chain length and functionalization, including the C1 compound formaldehyde. Therefore, disclosed herein are reaction pathways that convert the product of the HACL-catalyzed reaction, a 2-hydroxyacyl-CoA, to an aldehyde that can be further extended by formyl-CoA.

In some embodiments, the 2-hydroxyacyl-CoA is reduced to a 2-hydroxyaldehyde by an acyl-CoA reductase (ACR; E.C. 1.2.1.-, e.g. 1.2.1.10, 1.2.1.76, 1.2.1.84). Further ligation of the 2-hydroxyaldehydes with formyl-CoA by HACL give polyhydroxyacyl-CoAs and further polyhydroxyaldehydes, commonly known as aldoses. Polyhydroxyaldehydes can in principle serve as substrates of the HACL-catalyzed reaction, which is referred to herein as “aldose elongation.”

Further reduction of the 2-hydroxyaldehyde to give a 1.2-diol is possible by a suitable 1.2-diol oxidoreductase (DOR; E.C. 1.1.1.77) or alcohol dehydrogenase (ADH; E.C. 1.1.1.71). In some embodiments, the DOR is E. coli FucO.

Escherichia coli FucO is described in Pereira, B. et al. Metab. Eng. 34, 80-87 (2016), which incorporated by reference for the teaching of this enzyme. Bacteroides thetaiotaomicron RhaO is described in Patel, E. H., et al. Res. Microbiol. 159, 678-684 (2008), which incorporated by reference for the teaching of this enzyme. Clostridium sphenoides DOR is described in Tran-Din, K., et al. Arch. Microbiol. 142, 87-92 (1985), which incorporated by reference for the teaching of this enzyme. Microcyclus eburneus DOR is described in Kawagishi, T., et al. Agric. Biol. Chem. 44, 949-950 (1980), which incorporated by reference for the teaching of this enzyme. Paenibacillus macerans DOR is described in Weimer, P. J. Appl. Environ. Microbiol. 47, 263-267 (1984), which incorporated by reference for the teaching of this enzyme.

Dehydration of 1.2-diol can be catalyzed by the activity of diol dehydratase (DDR; E.C. 4.2.1.28) to give an aldehyde. Further elongation of the aldehyde by formyl-CoA, which is referred to herein as “aldehyde elongation,” results in the extension of an alkyl chain, analogous to the fatty acid biosynthesis or reverse β-oxidation pathways.

In some embodiments, a combination of the above routes can be implemented at the same time such that for some molecules, elongation takes place through aldose elongation, whereas for other molecules, elongation takes place through aldehyde elongation. Both routes can be simultaneously present at the same time in the same system.

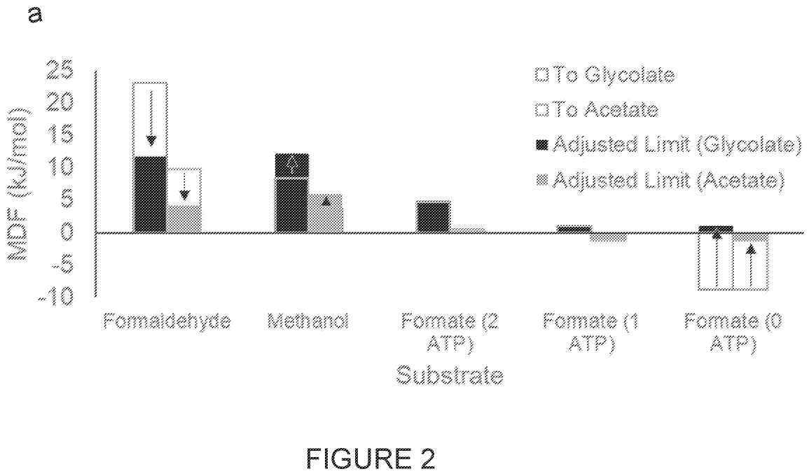

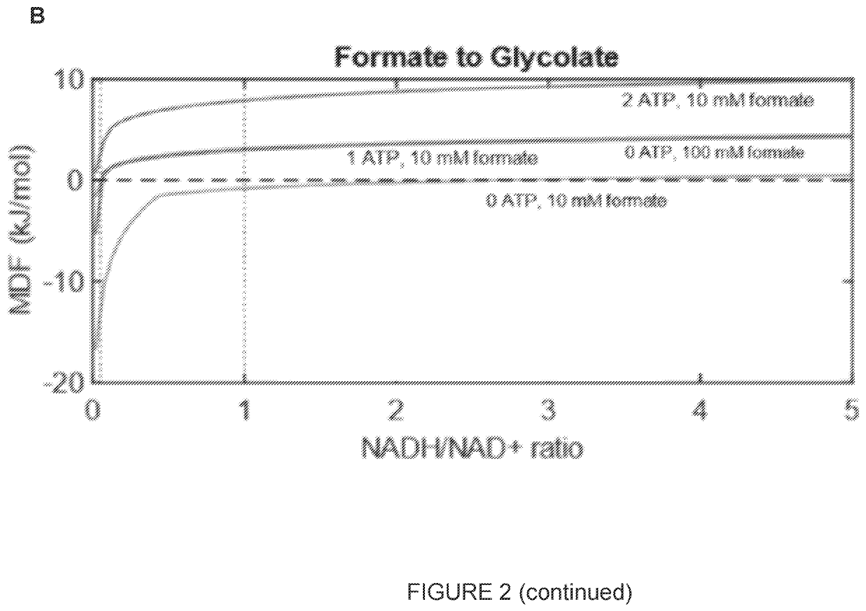

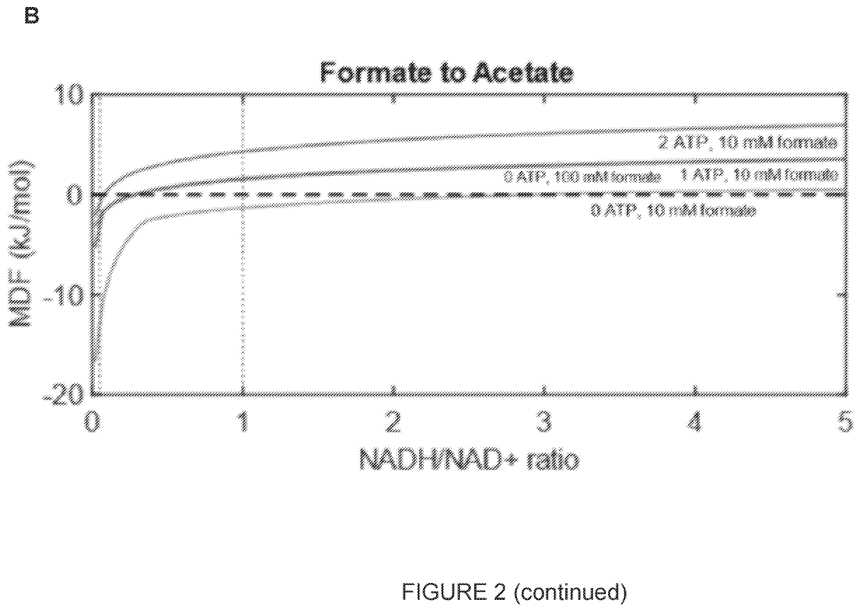

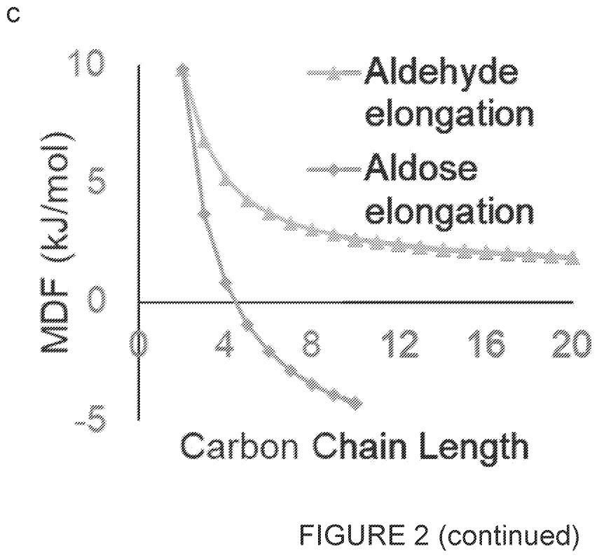

In some embodiments, intermediates of the above reactions serve as metabolites for the growth of the microorganism. In other embodiments, the intermediates of the above reactions serve as precursors to or are converted to growth substrates of the microorganism. Examples of these products are highlighted in FIG. 2 and include ketoacids, hydroxyacids, aldehydes, diols and polyols.

In some embodiments, the described pathways are provided within the context of a microbial host. In some embodiments, the microbial host is cultured in a fermentation system to produce the multi-carbon molecules. In other embodiments, a microbial system is used to produce the enzymes, which are then extracted from the microbes for use in a cell-free system. In other embodiments, the enzymes are produced separately and individual added to the system.

In some embodiments, the microbial system is comprised of more than one engineered microbial host, where the functions of C1 utilization, biomass production, and product synthesis are divided into multiple organisms, which are cultured in a fermentation system known as a coculture and wherein the overall result of the coculture is the conversion of C1 substrates into biomass and/or chemical products.

The pathway in a living system is generally made by transforming the microbe with one or more expression vector(s) containing a gene encoding one or more of the enzymes, but the genes can also be added to the chromosome by recombinant engineering, homologous recombination, gene editing, and similar techniques. Where the needed protein is endogenous, as is the case in some instances, it may suffice as is, but is usually overexpressed for better functionality and control over the level of active enzyme. In some embodiments, one or more, or all, such genes are under the control of an inducible promoter.

The enzymes can be added to the genome or via expression vectors, as desired. Preferably, multiple enzymes are expressed in one vector or multiple enzymes can be combined into one operon by adding the needed signals between coding regions. Further improvements can be had by overexpressing one or more, or even all of the enzymes, e.g., by adding extra copies to the cell via plasmid or other vector. Initial experiments may employ expression plasmids hosting 3 or more ORFs for convenience, but it may be preferred to insert operons or individual genes into the genome for stability reasons.

Still further improvements in yield can be had by reducing or suppressing competing pathways, such as those pathways for making e.g., acetate, formate, ethanol, and lactate, and it is already well known in the art how to reduce or knockout these pathways. See e.g., U.S. Pat. Nos. 7,569,380, 7,262,046, 8,962,272, 8,795,991, 8,129,157, and 8,691,552, each incorporated by reference herein in its entirety for all purposes. Many others have worked in this area as well.

Following the construction of a suitable strain containing the engineered pathway, culturing of the developed strains can be performed to evaluate the effectiveness of the pathway at its intended goal—the production of products from single carbon compounds. The organism can be cultured in a suitable growth medium, and can be evaluated for product formation on single carbon substrates, from methane to CO₂, either alone or in combination with multi-carbon molecules. The amount of products produced by the organism can be measured by ultra performance liquid chromatograph (UPLC) or gas chromatography (GC), and indicators of performance such as growth rate, productivity, titer, yield, or carbon efficiency can be determined.

Further evaluation of the interaction of the pathway enzymes with each other and with the host system can allow for the optimization of pathway performance and minimization of deleterious effects. Because the pathway is under synthetic control, rather than under the organism's natively evolved regulatory mechanisms, the expression of the pathway is usually manually tuned to avoid potential issues that slow cell growth or production and to optimize production of desired compounds.

Additionally, an imbalance in relative enzyme activities might restrict overall carbon flux throughout the pathway, leading to suboptimal production rates and the buildup of pathway intermediates, which can inhibit pathway enzymes or be cytotoxic. Analysis of the cell cultures by high performance liquid chromatography (HPLC) or GC can reveal the metabolic intermediates produced by the constructed strains. This information can point to potential pathway issues.

As an alternative to the in vivo expression of the pathway, a cell free in vitro version of the pathway can be constructed. By purifying the relevant enzyme for each reaction step, the overall pathway can be assembled by combining the necessary enzymes in a reaction mixture. With the addition of the relevant cofactors and substrates, the pathway can be assessed for its performance independently of a host.

In some embodiments of, single carbon molecules, such as carbon dioxide, formate, formaldehyde, methanol, methane, and carbon monoxide are solely used in the production of products containing at least one carboxyl group. In this embodiment, both formaldehyde and formyl-CoA, are produced from single carbon molecules as described earlier.

General methods for gene synthesis and DNA cloning, as well as vector and plasmid construction, are well known in the art, and are described in a number of publications. More specifically, techniques such as digestion and ligation-based cloning, as well as in vitro and in vivo recombination methods, can be used to assemble DNA fragments encoding a polypeptide that catalyzes a substrate to product conversion into a suitable vector. These methods include restriction digest cloning, sequence- and ligation-independent Cloning (SLIC), Golden Gate cloning, Gibson assembly, and the like. Some of these methods can be automated and miniaturized for high-throughput applications.

Gene cassettes for expressing an engineered metabolic pathway in a host microorganism are known in the art. The cassette can comprise one or more open reading frames (ORFs) which encode the enzymes of the introduced pathway, a promoter for directing transcription of the downstream ORF(s) within the operon, ribosome binding sites for directing translation of the mRNAs encoded by the individual ORF(s), and a transcriptional terminator sequence. Due to the modular nature of the various components of the expression cassette, one can create combinatorial permutations of these arrangements by substituting different components at one or more of the positions. One can also reverse the orientation of one or more of the ORFs to determine whether any of these alternate orientations improve the product yield.

In some embodiments, the host microorganism for expressing the plasmid is a methanotroph, and plasmid vector(s) containing the metabolic pathway expression cassettes are mobilized into these organisms via conjugation.

In an alternative method for expressing metabolic pathway genes in a microbial host, the biosynthetic pathway genes can be inserted directly into the chromosome. Methods for chromosomal modification include both non-targeted and targeted deletions and insertions.

In some embodiments, the disclosed systems and methods also involve recovering and purifying the desired product from the fermentation broth. The method to be used depends on the physico-chemical properties of the product and the nature and composition of the fermentation medium and cells. For example, U.S. Pat. No. 8,101,808 describes methods for recovering C3-C6 alcohols from fermentation broth using continuous flash evaporation and phase separation processing. In some embodiments, solids may be removed from the fermentation medium by centrifugation, filtration, decantation. In some embodiments, the multi-carbon compounds are isolated from the fermentation medium using methods such as distillation, azeotropic distillation, liquid-liquid extraction, adsorption, gas stripping, membrane evaporation, or pervaporation.

For longer-chain alcohols, such as fatty alcohols, U.S. Pat. No. 8,268,599 describes methods for separating these components from the aqueous phase of the fermentation by bi-phasic separation, whereby the immiscibility of the product compounds with the fermentation broth allows the organic phase to be collected and removed. This separation can also reduce the toxic effects of the product on the host microbial cells. U.S. Publication No. 2007/0251141 describes methods for recovering fatty acid methyl esters (FAMEs) from a liquid suspension by adding urea and creating a phase separation whereby the saturated and unsaturated FAMEs can be recovered separately. Membrane separation methods can also be applied to purifying fatty acid ester products such as biodiesel.

In certain embodiments, a methane substrate of the invention is provided or obtained from a natural gas source, wherein the natural gas is “wet” natural gas or “dry” natural gas. Natural gas is referred to as “dry” natural gas when it is almost pure methane, having had most of the other commonly associated hydrocarbons removed. When other hydrocarbons are present, the natural gas is referred to as “wet”. Wet natural gas typically comprises about 70-90% methane, about 0-20% ethane, propane and butane (combined total), about 0-8% C02, about 0-5% N2, about 0-5% H2S and trace amounts of oxygen, helium, argon, neon and xenon. In certain other embodiments, a methane substrate of the invention is provided or obtained from methane emissions, or methane off-gases, which are generated by a variety of natural and human-influenced processes, including anaerobic decomposition in solid waste landfills, enteric fermentation in ruminant animals, organic solids decomposition in digesters and wastewater treatment operations, and methane leakage in fossil fuel recovery, transport, and processing systems.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

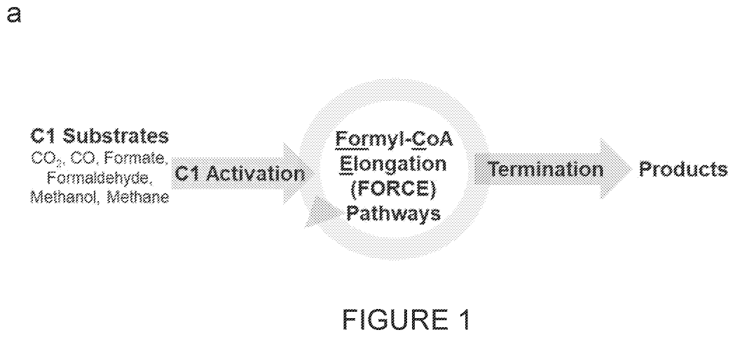

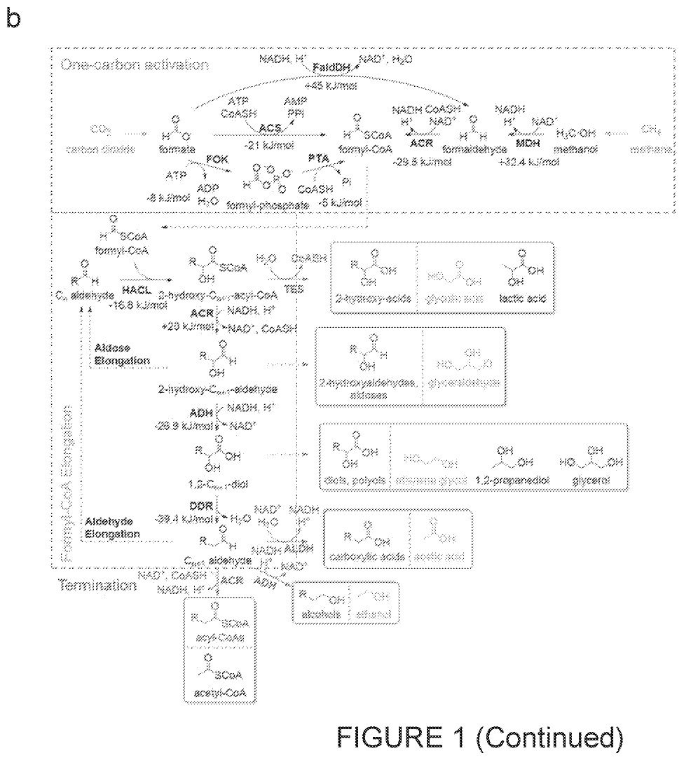

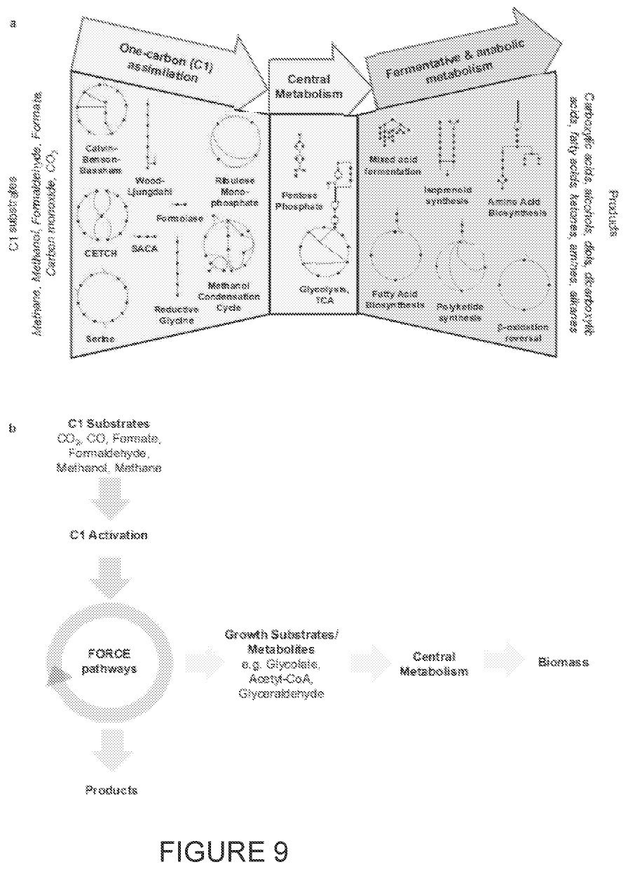

This Example investigates an alternative to canonical C1 metabolism that is orthogonal (Pandit, A V., et al R. Nat. Commun. 8:1-11 (2017)) to the central metabolic processes of the host organism and that is based on the newly discovered application of formyl-CoA as a C1 elongation unit by HACL. Rather than the “bowtie” architecture of metabolism (FIG. 9A), in which central metabolic processes are the major branch point between product synthesis and growth, the orthogonal architecture allows for product synthesis independently from the host central and product synthesis pathways (FIG. 1A). This type of metabolic architecture relies on the ability to produce carbon skeletons necessary for diverse product synthesis directly from C1 substrates. This Example reports conceptualization and design of biochemical pathways enabling this orthogonal architecture, based on formyl-CoA elongation (FORCE) pathways, provide analysis of their feasibility and performance, and demonstrate their function in prototype systems. FORCE pathways can serve as the basis for both bioproduct synthesis and C1-trophy via the production of growth substrates native to the microbial host organism. This this example provides systems comprising a one carbon substrate capable of growing microorganisms that were not previously able to grow on such medium. This growth on C1 substrates is novel and was by system design utilizing the one carbon substrate as the only energy source. |FIGS. 7-9, 14, and 16-18 (and material that relates to these figures) described in detail growth on C1 substrates.

Results

Design of an Orthogonal Metabolic Architecture Based on C1 Utilization and Product Synthesis

The orthogonal metabolic architecture developed here has three primary features 1) activation of C1 substrates into a suitable building block for carbon chain elongation; 2) iterative elongation of a carbon chain by one carbon per cycle; and 3) termination of the pathway resulting in accumulation of the product of interest. Based on our previous findings (Chou, A., et al. Nat. Chem. Biol. 15:900-906 (2019)), whether a design conceived and implemented based on the use of formyl-CoA was developed.

The role of formyl-CoA in metabolism is most well-established in the degradation of multi-carbon compounds and reports of the generation of formyl-CoA from C1 molecules are sparse. Acyl-CoAs, though, are a convenient intermediate between the carboxylate and aldehyde forms enabling formyl-CoA generation from both oxidized and reduced C1 substrates (FIG. 1b: one-carbon activation panel). From formaldehyde, formyl-CoA can be produced via acyl-CoA reductase (ACR)¹⁶ activity, and methanol oxidation to formaldehyde by methanol dehydrogenase (MDH) has been the subject of numerous studies¹⁸⁻²⁰. Formyl-CoA may be produced from formate by CoA transferases²¹ or CoA ligases, such as the promiscuous activity Escherichia coli acetyl-CoA synthetase (EcACS)⁶. While the latter is AMP forming (consuming 2 ATP equivalents), evidence of an ADP forming route exists via the intermediate formyl-phosphate through formate kinase (FOK) and phosphotransacylase (PTA)²². ATP-independent conversion of formate to formyl-CoA via reduction of formate to formaldehyde by formaldehyde dehydrogenase (FaIdDH) is also possible²³, albeit thermodynamically challenging (FIG. 1b). Furthermore, CO₂ can be converted to formate by the reverse activity of formate dehydrogenase (or carbon dioxide reductase)^{24,25} and methane to methanol by methane monooxygenase²⁶, which when coupled to the reactions described above can lead to formyl-CoA formation.

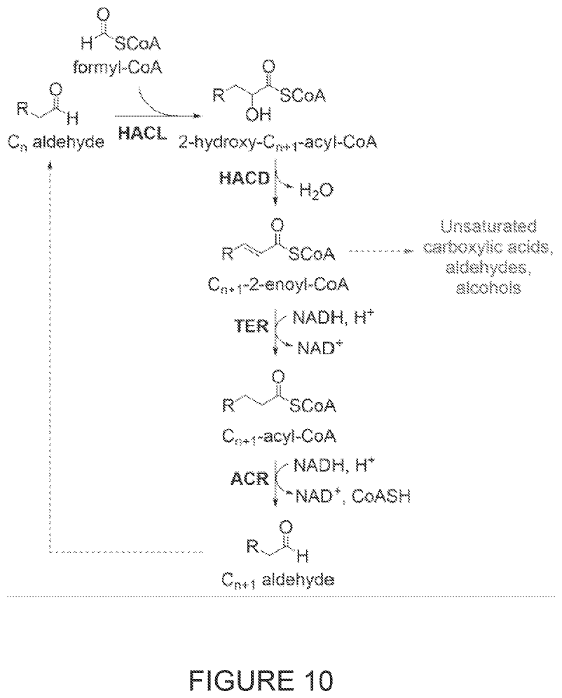

The orthogonal, de novo construction of diverse carbon skeletons by C1 elongation necessitates an iterative pathway similar to those found in nature that construct carbon skeletons from C2-C5 metabolites²⁷, yet existing outside of central metabolism. Because 2-hydroxyacyl-CoA lyase (HACL) has broad carbon chain length specificity¹⁶, it is a good candidate for establishing an iterative pathway. We evaluated reaction pathways potentially enabling iteration by converting the product of the HACL-catalyzed reaction, 2-hydroxyacyl-CoA, to an aldehyde that can be further extended by formyl-CoA. At the α-carbon, dehydration is possible, transforming the 2-hydroxyacyl-CoA to a 2-enoyl-CoA²⁸ (FIG. 10) similar to the well-established acrylate pathway²⁹. 2-enoyl-CoA generation is also convenient as these intermediates are involved in β-oxidation, potentially allowing the use of the enzymatic toolkit and knowledge established for the β-oxidation reversal platform³⁰⁻³². Dehydration of 2-hydroxyacyl-CoA, however, is much more challenging than dehydration of 3-hydroxyacyl-CoA, thus requiring an oxygen-sensitive radical mechanism³³. It also requires the existence of a β-carbon thus restricting pathway implementation to intermediates 3 carbons or larger.

Due to these issues, we investigated transformations of the thioester. Reduction of the CoA-thioester gives a 2-hydroxyaldehyde (FIG. 1b: formyl-CoA elongation panel), which is possible due to the non-specific activity of certain acyl-CoA reductases (ACRs)¹⁶. Ligation of 2-hydroxyaldehydes with formyl-CoA by HACL gives polyhydroxyacyl-CoAs and further polyhydroxyaldehydes, commonly known as aldoses. Polyhydroxyaldehydes can in principle serve as substrates of the HACL-catalyzed reaction, which we refer to as aldose elongation (FIG. 1b: formyl-CoA elongation panel).

Reduction of the 2-hydroxyaldehyde via diol oxidoreductase (DOR) activity to give a 1.2-diol is also possible. For example, E. coli FucO catalyzes the interconversion of 1.2-diols with 2-hydroxyaldehydes³⁴. 1_2-diol dehydration to an aldehyde can be catalyzed by the activity of diol dehydratase (DDR), effectively accomplishing α-reduction. While diol dehydration also requires a radical mechanism, the B12-dependent DDR is oxygen tolerant and has been the subject of numerous protein and metabolic engineering studies³⁵⁻³⁷. Elongation of this aldehyde by formyl-CoA, which we refer to as aldehyde elongation, enables extension of an alkyl chain, analogous to the two-carbon elongation in fatty acid biosynthesis³⁸ or reverse β-oxidation³⁹ pathways. We collectively refer to these pathways (aldose elongation, α-reduction, and aldehyde elongation) as formyl-CoA elongation (FORCE) pathways, as they facilitate the use of formyl-CoA as a carbon chain elongation unit (FIG. 1b: formyl-CoA elongation panel).

Various product classes can be produced as intermediates or from derivatives of intermediates of FORCE pathways (FIG. 1b), some of which also support microbial growth (FIG. 9). Aldose sugars, for example, are a direct result of the 2-hydroxyaldehyde node. Diols, including major industrial chemicals such as ethylene glycol, are a result of the 1.2-diol node. Derivatives of the 2-hydroxyacyl-CoA node include 2-hydroxyacids, such as industrial products glycolic and lactic acids, produced by a thioesterase catalyzed reaction. Numerous chemical classes can be derived from the aldehyde node⁴⁰, including carboxylic acids, alcohols, and acyl-CoAs that can serve as precursors of other products.

### Thermodynamic Analysis of FORCE Pathways for C1 Utilization

The standard Gibbs free energies of the pathway reactions (FIG. 1b) make readily apparent the potential challenging reactions, but a holistic analysis of pathway thermodynamics is necessary that considers the ability for reactions to influence each other. For this, we applied the “Max-min Driving Force (MDF)” approach.⁴¹.

We evaluated the MDF of the FORCE pathways to produce C2 metabolites glycolate and acetate from C1 substrates. Only soluble C1 substrates were evaluated as mass transport limitations can significantly limit CO₂ and methane utilization and are outside the scope of this analysis. The representative C2 products, glycolate and acetate, are both pathway products and growth substrates, with glycolate requiring the shortest pathway and acetate requiring the entire sequence of aldehyde elongation reactions. As shown in FIG. 2a, there is a greater driving force toward glycolate production than acetate for each substrate. This results from the thermodynamically favorable hydrolysis of glycolyl-CoA, whereas acetate production requires the thermodynamically challenging reduction of glycolyl-CoA. Using standard limits of metabolite concentrations⁴¹, formaldehyde allows the greatest MDF as it does not require NAD⁺-dependent methanol oxidation or formyl-CoA reduction to formaldehyde. However, despite the thermodynamic challenge of methanol oxidation, there is sufficient driving force in the desired direction for the net production of glycolate and acetate. The driving force for formate utilization is the lowest. Here, ATP hydrolysis assists in the activation of formate. The hydrolysis of 2 ATP equivalents provides just enough driving force for the net production of acetate, while the utilization of 1 ATP equivalent only provides enough driving force for glycolate production. As expected, the ATP-independent route is not feasible under these conditions.

While the above analysis assumes a standard constraint on metabolite concentrations (1 μM-10 mM⁴¹), we sought to apply realistic substrate concentrations based on physical limitations such as the toxicities of the C1 substrates (FIG. 2a). Although some organisms can survive at formaldehyde concentrations on the order of 10 mM⁴², the upper bound of formaldehyde was adjusted to a more reasonable 0.1 mM, resulting in a decrease of the MDFs. Increasing the upper bound of methanol, which has been used at concentrations on the order of 100 mM⁴³, led to larger MDFs. Interestingly, at these concentrations, the driving force for methanol utilization becomes greater than that for formaldehyde. Similarly, E. coli has the ability to grow in the presence of formate concentrations on the order of 100 mM^{9,44}. Increasing the bound on formate concentration had no effect on the MDF in the 1 or 2 ATP consumption scenarios, but it had a major impact on the MDF of the 0 ATP route, enabling the synthesis of glycolate.

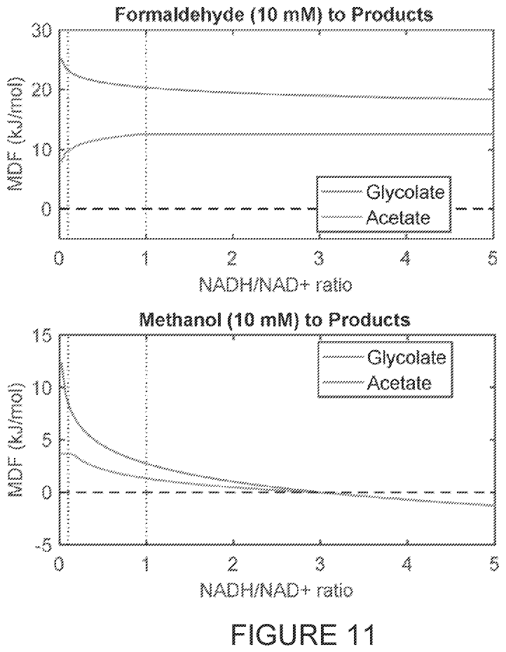

The NADH/NAD⁺ ratio was also a major constraint on pathway thermodynamics. While we initially used a constraint of 0.1⁴¹, reflecting growth of E. coli under aerobic conditions, the physiological NADH/NAD⁺ can vary, reaching values near or greater than 1 under anaerobic conditions⁴⁵⁻⁴⁷. In the physiological range (0.1-1), pathway driving forces remained positive for formaldehyde and methanol as substrates (FIG. 11). As expected, a low ratio was favorable when the pathway was redox generating (methanol to acetate/glycolate and formaldehyde to glycolate), with a high ratio favorable when the pathway was redox consuming (formate to acetate/glycolate) or redox balanced (formaldehyde to acetate—likely because the reduction reactions are more thermodynamically challenging). The NADH/NAD⁺ ratio is critical for the driving force of formate utilization pathways (FIG. 2b). Here, a NADH/NAD⁺ ratio at the higher end of the physiological range in combination with increasing the formate concentration to 100 mM enables a positive driving force for glycolate or acetate production even without ATP hydrolysis.

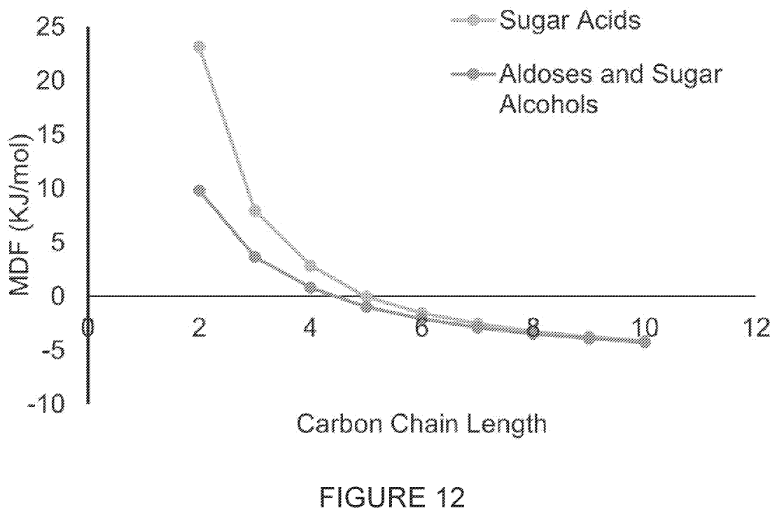

We further evaluated the ability of FORCE pathways to support iteration using formaldehyde as the exemplary substrate due to its intermediate redox state. The thermodynamics of both the aldose and aldehyde elongation pathways support iteration up to 4 carbons (FIG. 2c). After 4 carbons, the aldose elongation mode becomes unfavorable, likely due to the cumulative effect of successive acyl-CoA reduction reactions. The aldehyde elongation mode remains favorable despite requiring the same acyl-CoA reductions, likely due to the thermodynamically favorable reactions catalyzed by DOR and DDR. Different C1 activation and termination pathways have an influence on the MDF of the overall elongation cycles when the number of iterations is low. As the number of iterations increases, the thermodynamics of the elongation cycle reactions dominate (FIG. 12).

### In Vitro Pathway Validation

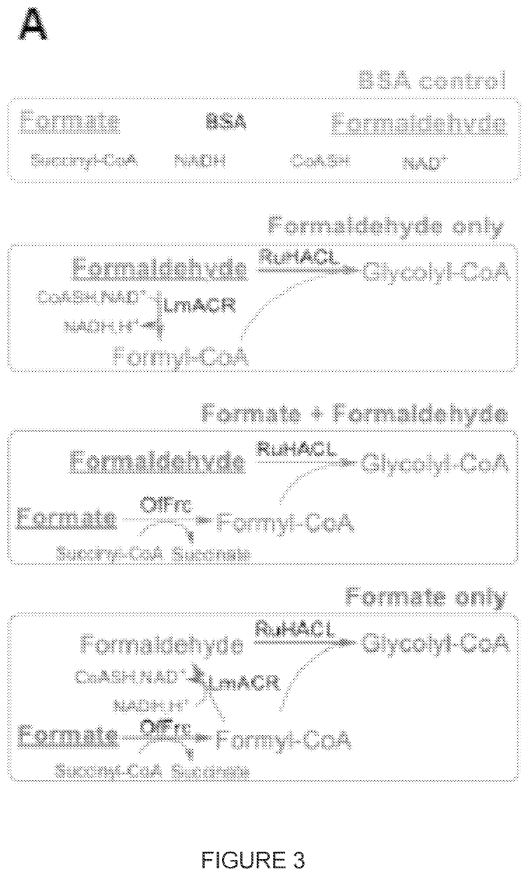

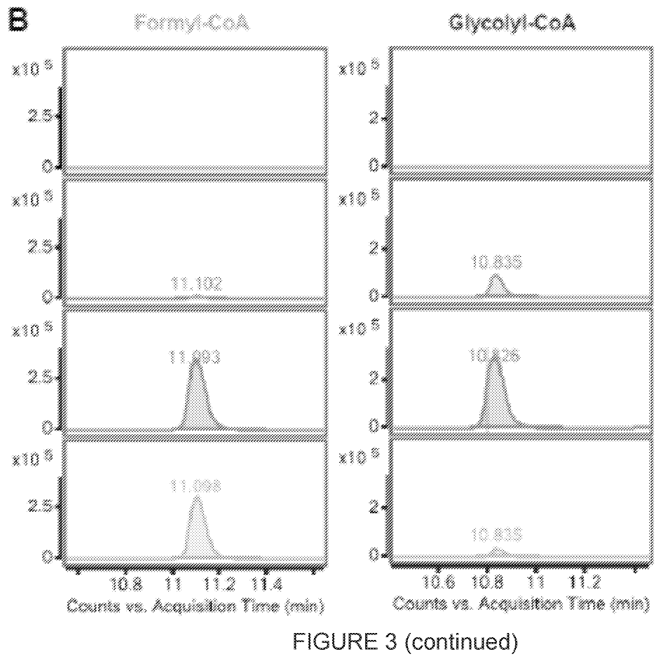

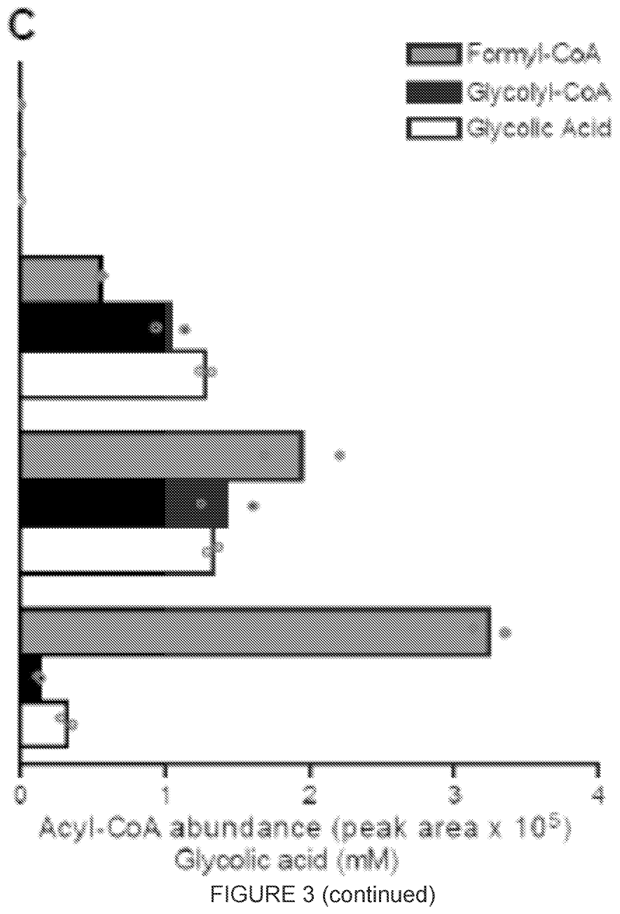

The key prerequisite to FORCE pathways is the generation of formyl-CoA and formaldehyde. To verify the function of these reactions, we developed purified enzyme systems to monitor formation of formyl-CoA and the HACL condensation product glycolyl-CoA from different C1 substrates (FIG. 3).

Since formyl-CoA can be produced from formaldehyde by an ACR, we observed the formation of both formyl-CoA and glycolyl-CoA in a reaction containing Listeria monocytogenes ACR (LmACR) and Rhodospirillales bacterium URHD0017 HACL (RuHACL)¹⁶ using formaldehyde as the only C1 substrate. Formyl-CoA can also be derived from oxidized C1 substrates by the activation of formate. Using a formyl-CoA transferase from Oxalobacter formigenes (OfFrc) and succinyl-CoA as the CoA donor, formate activation to formyl-CoA was observed, and, with the addition of formaldehyde, resulted in the formation of glycolyl-CoA. Using formate as sole substrate, formaldehyde was generated in situ by formyl-CoA reduction using LmACR, albeit with lower glycolyl-CoA production than when formaldehyde was added. Together these results suggest that in this enzyme system, the limitation is imposed by the ACR reaction either due to the activity of the enzyme or constraints due to the need for the appropriate form of NAD(H). In support of the latter hypothesis, in the oxidative direction (i.e. formaldehyde to formyl-CoA) the amount of glycolate observed following hydrolysis of the CoA thioesters was nearly equivalent to the 1 mM NADH added to the reaction. In the opposite direction, a less than equivalent amount of glycolate was observed, consistent with the thermodynamics of the reaction becoming unfavorable with decreasing NADH/NAD⁺ (FIG. 1b, 2b).

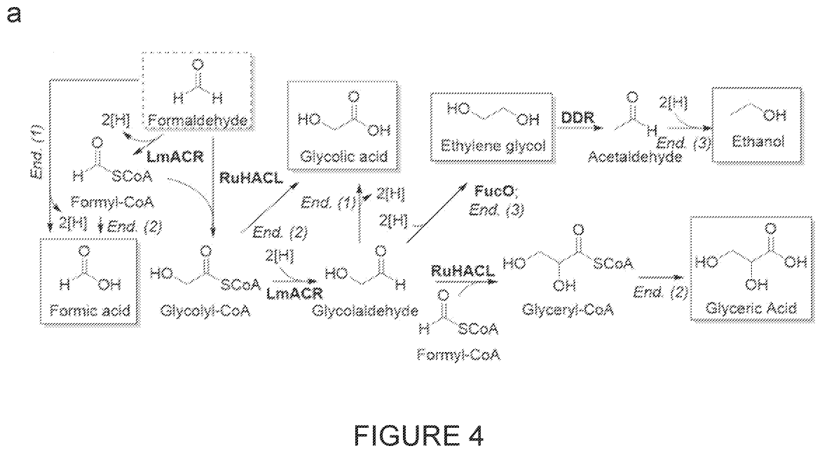

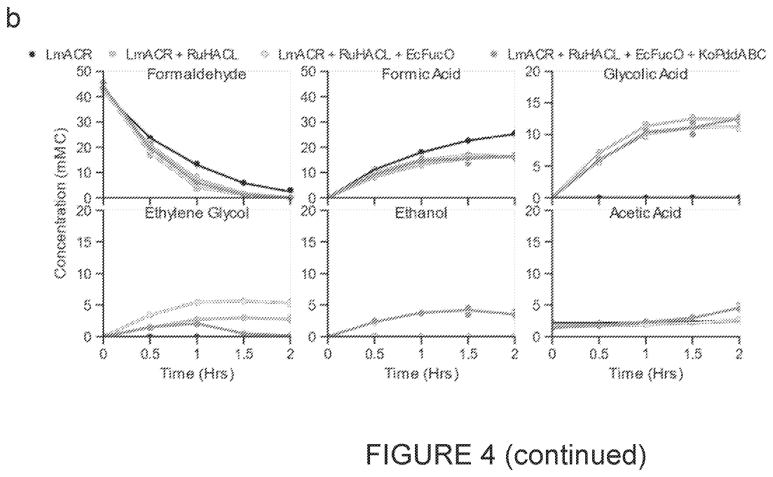

Having validated the core pathway reactions, cell-free metabolic engineering⁴⁸ was used to prototype FORCE pathways for product synthesis. Extracts of E. coli expressing each enzyme comprising the α-reductive FORCE pathway were successively combined, demonstrating pathway function in a stepwise manner (FIG. 4). Outside of the direct generation of 2-hydroxycarboxylates (e.g. glycolate) via thioester cleavage of the HACL generated 2-hydroxyacyl-CoA, other C2 products, as well as the aldose or aldehyde elongation pathways require reduction of this 2-hydroxyacyl-CoA (e.g. glycolyl-CoA for formaldehyde and formyl-CoA ligation) (FIG. 1b: formyl-CoA elongation panel). As we previously found that LmACR was able to act upon glycolaldehyde¹⁶, we used it to catalyze both formaldehyde oxidation to formyl-CoA and glycolyl-CoA reduction to glycolaldehyde (FIG. 4a). While LmACR alone resulted in only the conversion of formaldehyde to formate, inclusion of RuHACL resulted in glycolate production (FIG. 4b). Glycolaldehyde was not detected, possibly due to the presence of endogenous oxidoreductases in the cell extract system, which catalyzed its oxidation to glycolate (e.g. AldA, AldB, PuuC, PatD) or, to a lesser extent, reduction to ethylene glycol (e.g. FucO, YqhD, AdhP, EutG, and others⁴⁹).

The synthesis of the next reduction product, ethylene glycol (FIG. 4a), was significantly increased by the addition of a cell extract of E. coli overexpressing E. coli FucO, a 1.2-diol oxidoreductase (2-fold increase, from 1.37±0.1 mM to 2.73±0.03 mM) (FIG. 4b). Upon further addition of E. coli cell extract expressing Klebsiella oxytoca DDR, along with coenzyme B12, ethanol was detected (1.90±0.03 mM at one hour: FIG. 4b), likely due to the reduction of acetaldehyde (formed via ethylene glycol dehydration) by endogenous oxidoreductases, along with a corresponding decrease in ethylene glycol. At later time points an increase in acetate was observed, likely due to the oxidation of ethanol and acetaldehyde by endogenous oxidoreductase activity.

### In Vivo Implementation of FORCE Pathways

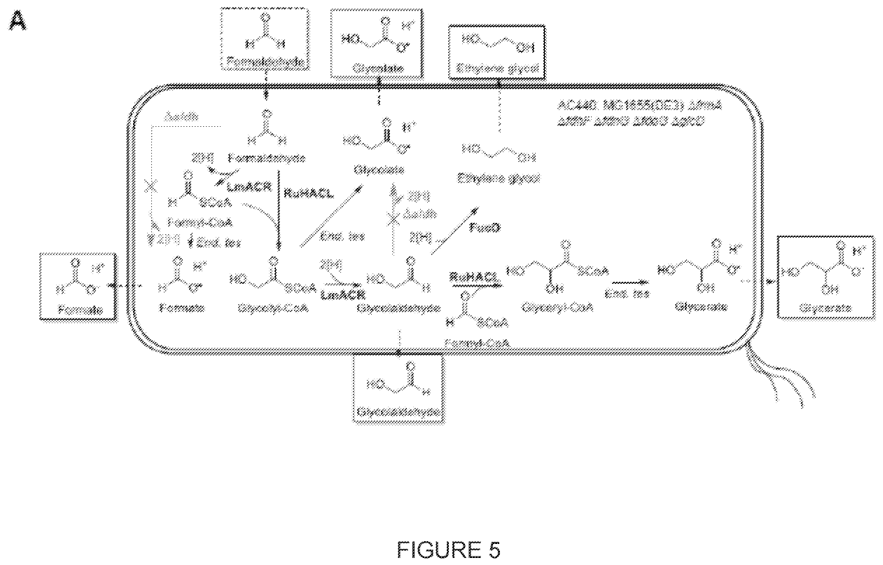

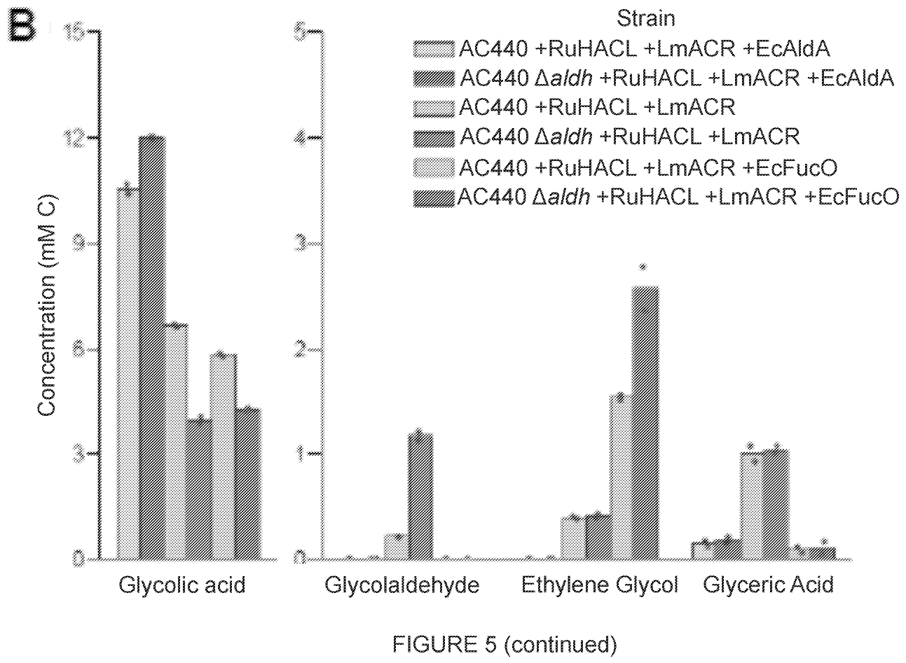

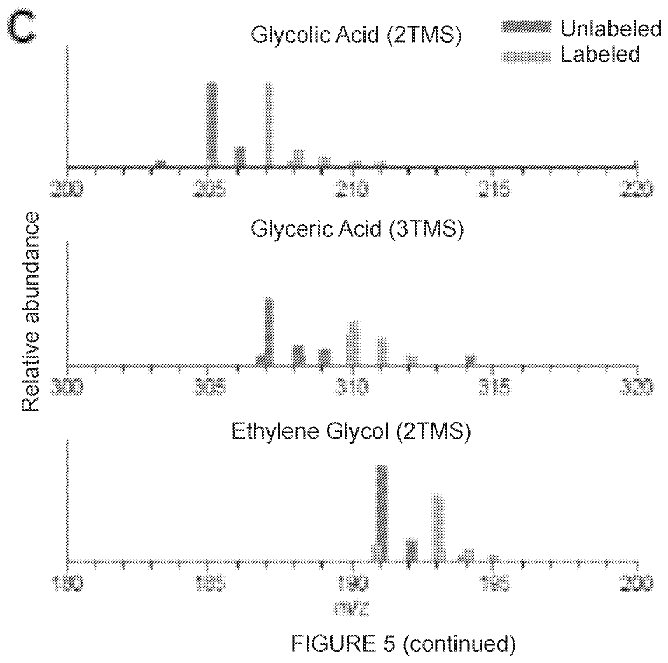

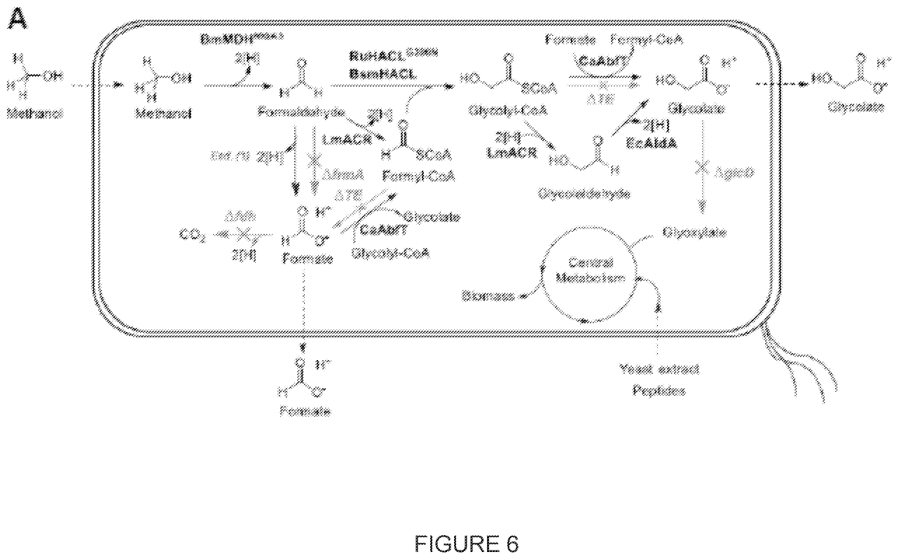

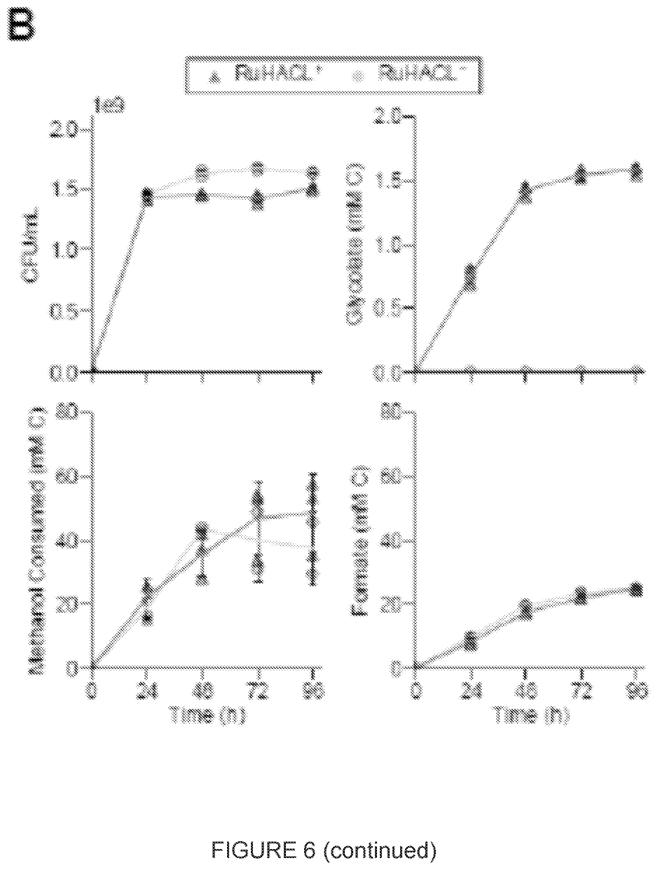

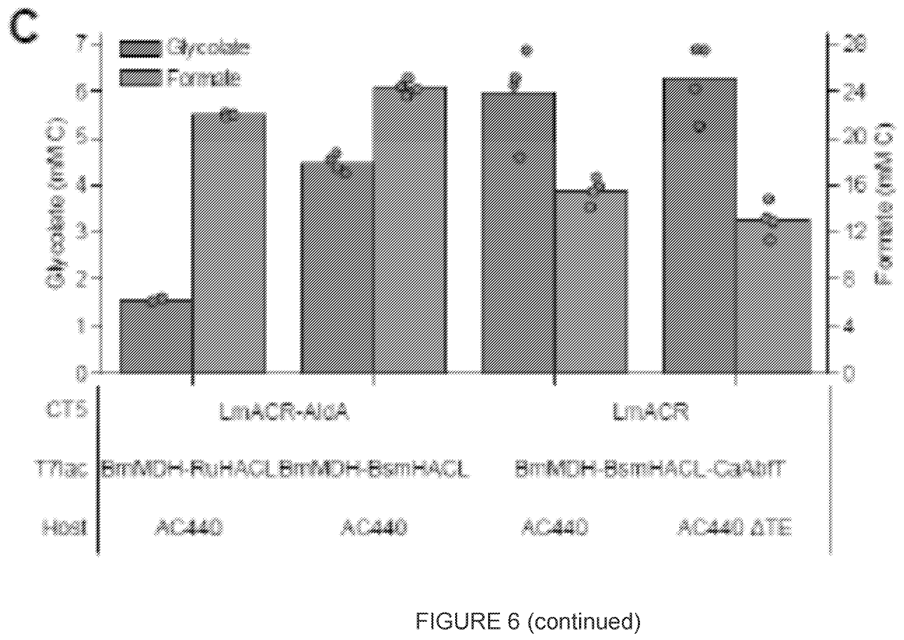

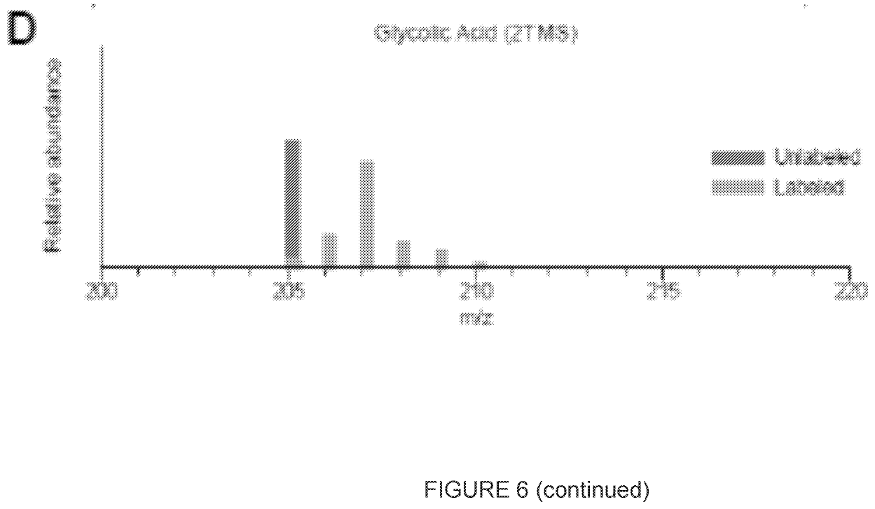

We sought to demonstrate key features of the designed platforms, as well as the synthesis of additional products and utilization of various C1 substrates using both resting and growing E. coli cultures (FIGS. 5 and 6). A key feature of the FORCE pathway design is iteration, which can be achieved through aldose or aldehyde elongation (FIG. 1b: formyl-CoA elongation panel, FIG. 2c). To demonstrate iterative aldose elongation in vivo we targeted the synthesis of three carbon product glycerate from formaldehyde (FIG. 5a). We started with a previously developed strain having C1 dissimilation and glycolate consumption knockouts (AC440: MG1655(DE3) ΔfrmA ΔfdhF ΔfdnG ΔfdoG ΔglcD) and overexpressing RuHACL^{G390N} LmACR, and EcAldA¹⁶. To promote glycolaldehyde accumulation and condensation with formyl-CoA, we removed EcAldA from the expression vector. While formaldehyde consumption was significantly reduced, accumulation of glycolaldehyde and glycerate was observed (FIG. 5b), demonstrating the iterative aldose elongation pathway. To increase the production of these compounds, we deleted genes encoding aldehyde dehydrogenases (ΔaldA ΔaldB ΔpatD ΔpuuC, collectively referred to as Δaldh), resulting in lower glycolate and higher glycolaldehyde when EcAldA was not overexpressed. However, these knockouts did not impact the accumulation of glycerate, perhaps indicating a limitation on the condensation reaction between glycolaldehyde and formyl-CoA catalyzed by RuHACL. We also extended the pathway to the next reduction product, ethylene glycol, by overexpressing E. coli fucO⁵⁰, which led to increased accumulation of ethylene glycol in the extracellular medium, with the Δaldh background further improving production (FIG. 5b). To verify that the observed products were derived from formaldehyde and not from residual multi-carbon substrates or biomass components, 13C-labeled formaldehyde was used as the substrate. Glycolic acid, ethylene glycol, and glyceric acid were found to be fully 13C labeled based on the characteristic [M−15]⁺ ions of the TMS derivatives of the products (FIG. 5c).

To extend the above established formaldehyde utilization pathway to methanol, we expressed a well-studied MDH variant from Bacillus methanolicus MGA3 (BmMDH2^{MGA3})¹⁸ for conversion of methanol to formaldehyde, in combination with RuHACL^{G390N}, LmACR, and EcAldA. Unlike formaldehyde, where toxicity necessitates the use of resting cells, methanol can also be directly added to growing E. coli cultures. When the engineered methanol utilizing strain was grown in the presence of complex nutrients and 500 mM methanol, glycolate formation was observed only in the strain expressing RuHACL (FIG. 6b). The conversion of methanol to glycolate by this strain was inefficient, however, with substantial accumulation of formate.

Seeking to improve performance, we replaced RuHACL^{G390N} with a newly identified HACL sourced from beach sand metagenome referred to here as BsmHACL (UniProt: A0A3C0TX30). BsmHACL increased glycolate accumulation about 3-fold (FIG. 6c). Despite improved glycolate production, formate accumulation remained high. In an effort to address this issue, the termination enzyme EcAldA was replaced with a CoA-transferase from Clostridium aminobutyricum (CaAbfT) previously found to have better properties than OfFrc⁵¹. CaAbfT serves to both release glycolate from glycolyl-CoA and reactivate formate to formyl-CoA for further condensation. When CaAbfT was expressed, glycolate accumulation increased around 33%, while formate accumulation was reduced by approximately 36%. Finally, with CaAbfT serving to terminate the pathway via the release of glycolate, endogenous thioesterases were not expected to be needed and were presumed to be in part responsible for the observed formate. Using a host strain deficient in thioesterases (ΔyciA ΔtesA ΔtesB ΔybgC Δydil ΔfadM), formate accumulation was further reduced. To verify that glycolate was derived from methanol, we used 13C-labeled methanol and observed that the [M−15]⁺ ion of the TMS derivative of glycolic acid was fully derived from 13C-methanol (FIG. 6d).

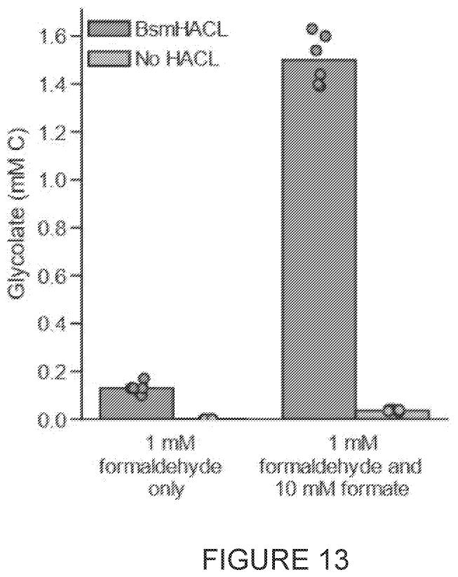

Having established CaAbfT as a promising route for formate activation, we evaluated whether CaAbfT could be used to incorporate exogenously supplied formate. Here, CaAbfT was expressed to activate formate without LmACR overexpression as no interconversion of formaldehyde and formyl-CoA is needed upon addition of formaldehyde. In the engineered strain expressing BsmHACL, a 12-fold increase in glycolate was observed when formate was included in the media compared to when formaldehyde was supplied alone (FIG. 13) with the total carbon accumulated as glycolate greater than the amount originally added as formaldehyde.

### Flux Balance Analysis of Synthetic Methylotrophy

Having demonstrated FORCE pathways for direct product synthesis some of which (e.g. glycolate, glycerate, acetate) can serve as growth substrates, their ability to enable synthetic methylotrophy in E. coli was evaluated in silico. Using a previously developed genome scale model of E. coli, iML1515⁵², we added reactions to the model comprising select pathways reported or proposed to enable methylotrophy. All pathways were evaluated with the reactions enabling the interconversion of C1 molecules at different reduction levels present. The full reactions implementing each pathway are shown in Table 3.

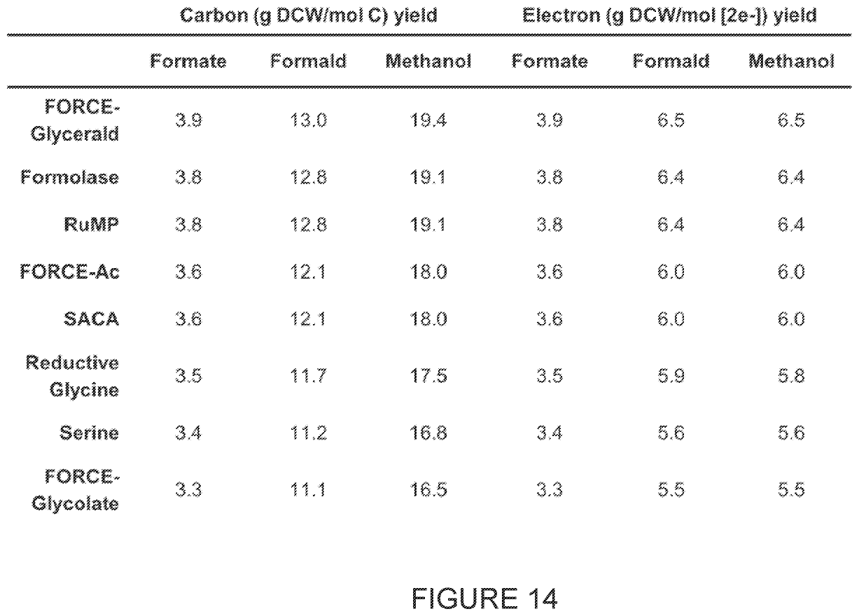

The simulation results suggest that all pathways previously proposed to enable some form of methylotrophy in E. coli, both natural (ribulose monophosphate or RuMP, serine) and synthetic (formolase, Synthetic Acetyl-CoA or SACA, reductive glycine),^{8,53,54}, are able to do so (FIG. 14). The FORCE pathways evaluated for the conversion of non-native C1 substrates to native growth substrates glycolate, acetate, and glyceraldehyde were no exception. This demonstrates another advantage of the platform's orthogonality, as direct route(s) to compound(s) representing physiological substrates for E. coli, or any other organism, enables FORCE pathways to be integrated at varying or multiple metabolic nodes to capitalize on native metabolism and regulation of substrate(s) utilization, opposed to needing to engineer them. An analysis of the flux distributions of the three modeled FORCE pathways provides further insights (FIG. 7). The FORCE pathway leading to the formation of glycolate utilizes a carbon-inefficient glycolate utilization pathway present in E. coli, which requires the decarboxylating condensation of two molecules of glyoxylate (FIG. 7a)⁵⁵. As such, production of more reduced C2 metabolites, such as glycolaldehyde or acetate, is preferred to glycolate as growth substrate. The predicted metabolism of glycolaldehyde is particularly interesting, as the model suggests a route for glycolaldehyde assimilation involving condensation with glycine and a reverse pyridoxal-5-phosphate biosynthesis pathway, ultimately resulting in pentose phosphate rearrangements to give glyceraldehyde-3-phosphate (FIG. 7b). This route appears to be preferred to the assimilation of acetyl-CoA via the glyoxylate bypass based on the predicted flux distribution. Direct production of glyceraldehyde from the HACL-based pathway results in the conversion of glyceraldehyde to glycerol, followed by native glycerol metabolism (FIG. 7c). As a result, pathways that lead to C3 molecules such as glyceraldehyde or dihydroxyacetone can take advantage of glycolytic reactions for the net production of ATP, ultimately enabling greater biomass yield. FORCE pathways also had promising characteristics based on other metrics such as redox balance, ATP requirements, and number of reactions required (Table 4).

### Two-Strain Co-Culture System to Evaluate Synthetic Methylotrophy

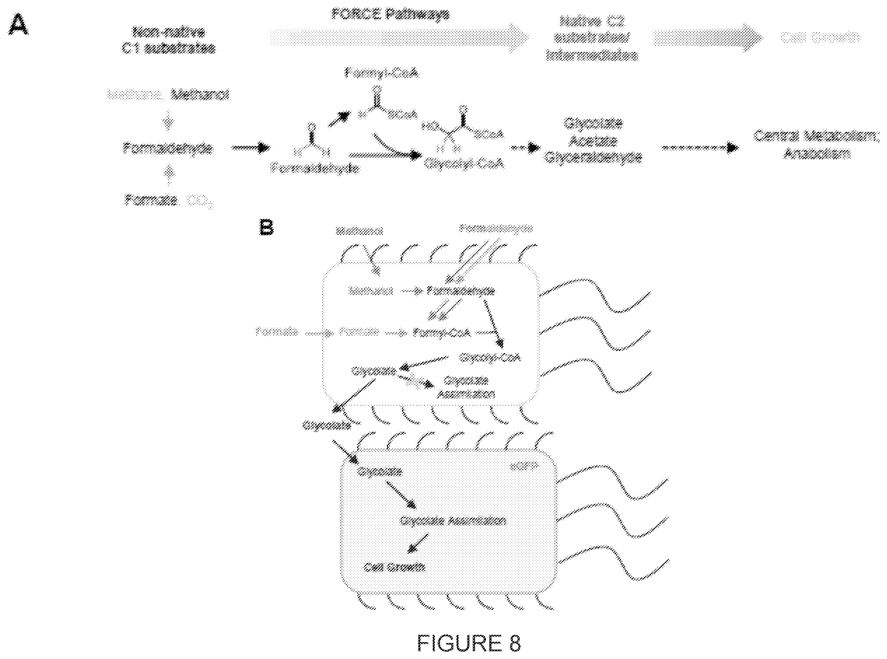

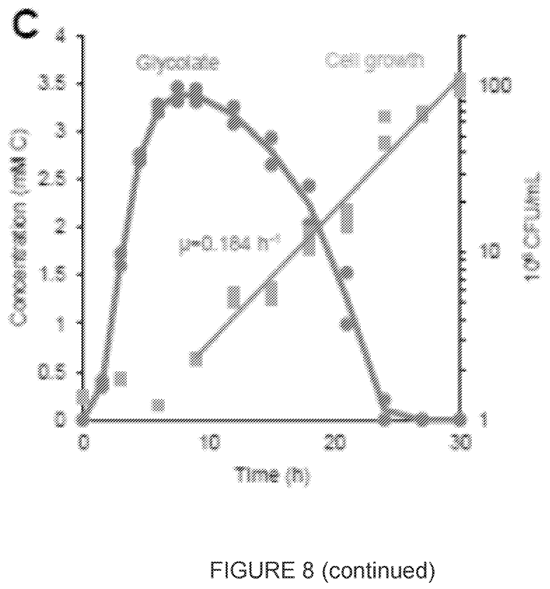

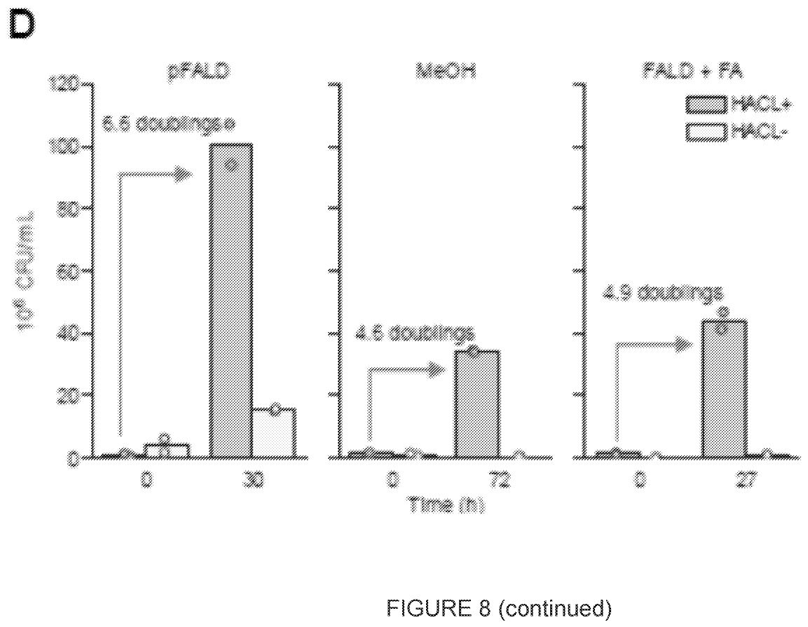

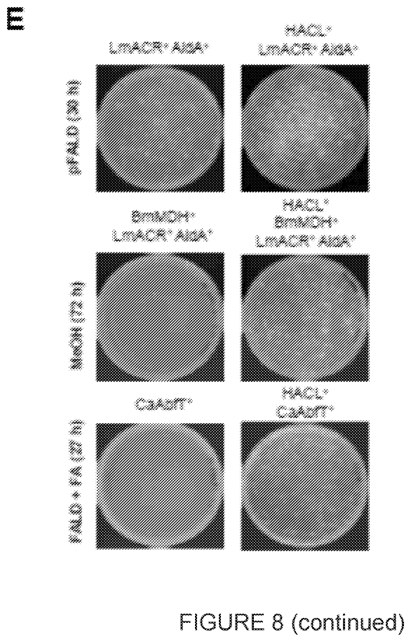

The orthogonality of FORCE pathways to metabolism allows full decoupling of the C1 conversion pathway from growth. This enables unique designs to evaluate the methylotrophic potential of the pathway (FIG. 8a; FIG. 9b). One potentially advantageous implementation might employ division of labor by separating multi-carbon compound generation and cell growth into two hosts, which would not be possible if the pathway directly interfaced with central metabolism, for example via aldose phosphates or acetyl-CoA, two common products of C1 assimilation pathways. Using this concept, we evaluated the ability for FORCE pathways to support E. coli growth on C1 substrates formaldehyde, formate, and methanol.

A two-strain E. coli system was designed and constructed to work in co-culture (FIG. 8b). The first strain, referred to as the producer strain, contained constructs to express the FORCE pathway for conversion of C1 substrates to the native C2 growth substrate glycolate but was deficient in the ability to consume glycolate. The second strain, referred to as the sensor strain, retained the ability to grow on glycolate and additionally constitutively expressed eGFP as a signal but did not express the FORCE pathway for glycolate production. These strains could thus be differentiated by both selection on glycolate minimal media plates and by detection of fluorescent colonies. To assess the feasibility of different substrates, three different producer strains were devised: for formaldehyde utilization, the producer strain expressed LmACR, BsmHACL, and EcAIdA; for evaluating formate utilization with formaldehyde, BsmHACL was expressed with CaAbfT; and for methanol utilization, a thioesterase deficient background expressing BmMdh^{MGA3}, LmACR, BsmHACL, and CaAbfT (FIG. 8b) was utilized.

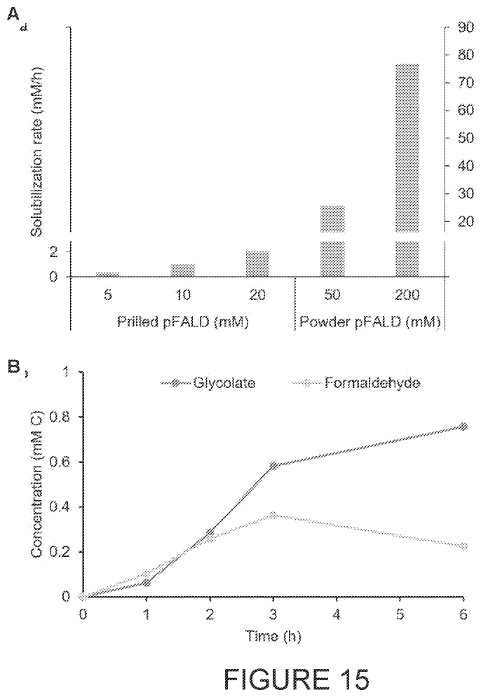

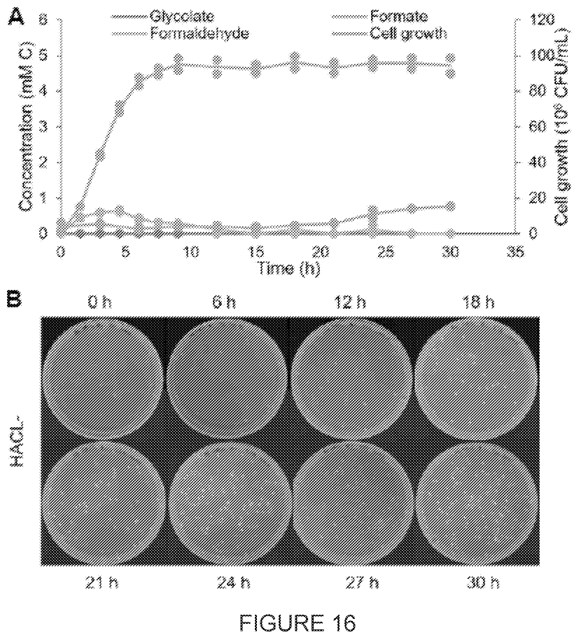

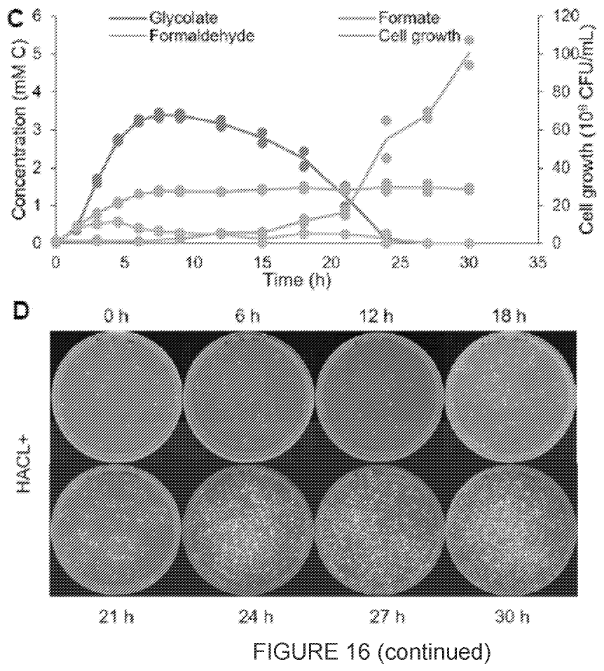

To enable growth conditions with formaldehyde, paraformaldehyde was used. Paraformaldehyde gradually depolymerizes to formaldehyde in aqueous media, with control over the solubilization rate through the selection of particle size and concentration (FIG. 15a). This enabled a system where formaldehyde could be kept at sub-millimolar concentrations, avoiding accumulation to toxic levels, with significant glycolate production still observed (FIG. 15b). In minimal media with (para)formaldehyde (the equivalent of 5 mM) as the sole carbon substrate, growth of the sensor strain was observed as indicated by the increase in colony-forming units (CFUs) relative to a control system in which the producer strain did not express BsmHACL (FIG. 8c, FIG. 16). Glycolate accumulated rapidly in the first 8 hours with sustained exponential growth of the sensor strain occurring after an initial lag phase. The sensor strain was found to have undergone around 6.6 doublings in 30 hours.

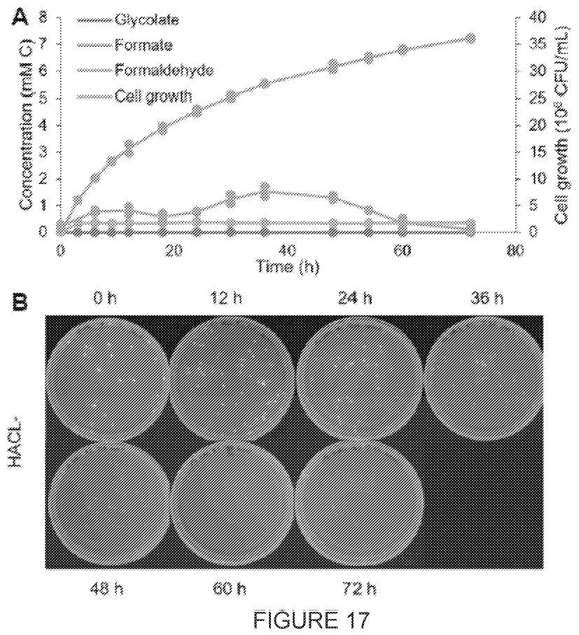

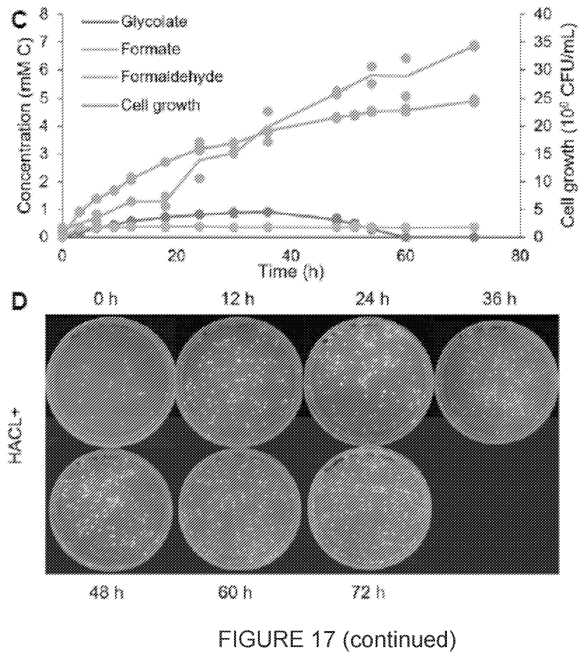

With methanol, growth of the sensor strain was observed only when the producer strain expressed BsmHACL (FIG. 8d, FIG. 17), however compared to the case for paraformaldehyde utilization the growth kinetics of the sensor strain differed, reflecting an approximately linear increase in CFUs over time. The difference in observed dynamics might reflect the limitation imposed by the rate of glycolate production from methanol by the producer strain, analogous to the phenomenon observed in constant feed-rate fed-batch culture⁵⁶. The utilization of methanol was substantially slower than the utilization of (para)formaldehyde, resulting in approximately 4.6 doublings in 72 hours.

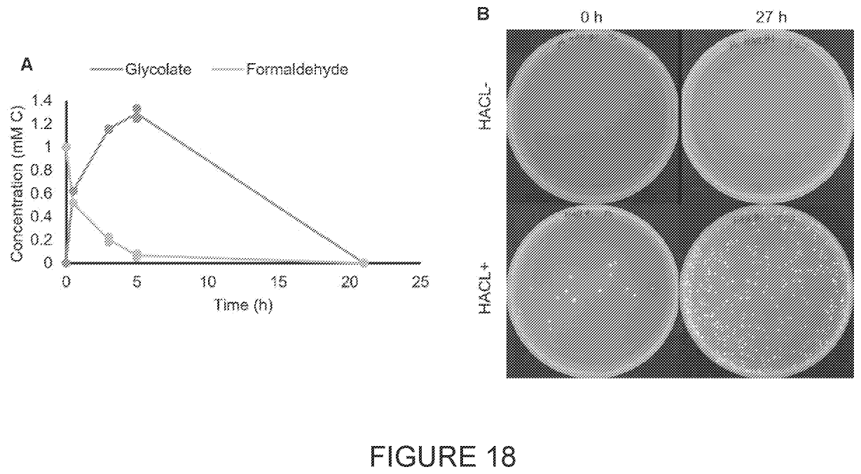

A similar experiment was performed using 1 mM formaldehyde and 10 mM formate co-substrate system. Here, more carbon was observed in glycolate than was added as formaldehyde, indicating the incorporation of formate (FIG. 18). Growth of the sensor strain was faster than growth on methanol but did not result in as many doublings as on 5 mM (para)formaldehyde. In 27 hours, around 4.9 doublings were observed (FIG. 8d).

### DISCUSSION

In the canonical architecture of metabolism, substrates are funneled into central metabolism with biosynthetic building blocks and products of interest derived from the resulting central metabolites. To date, attempts to engineer C1 bioconversion, even those exploiting synthetic pathways⁵⁻⁹ or novel enzyme designs⁶, have relied on central carbon metabolism. These designs, which exhibit minimal orthogonality, require optimizing a host's metabolic network to accommodate C1 bioconversion, which has proven challenging.

In this work, we present the design, analysis, and implementation of formyl-CoA elongation (FORCE) pathways, enabling C1 utilization and bioconversion in a manner orthogonal to the host metabolism. FORCE pathways are based on using formyl-CoA as an anabolic metabolite, which is enabled by 2-hydroxyacyl-CoA lyase (HACL) catalyzed acyloin condensation between formyl-CoA and carbonyl-containing substrates. Product synthesis is achieved with relatively high orthogonality to central metabolism compared to other approaches. Our thermodynamic analysis suggested favorable driving forces for FORCE pathway conversions of formate, formaldehyde, and methanol to glycolate or acetate as exemplary products. We demonstrate the potential of the self-contained, orthogonal pathway in both in vitro (purified enzymes and cell extracts) and in vivo (resting and growing cells) implementations, in which products of diverse functionality (e.g. glycolate, glycolaldehyde, ethylene glycol, ethanol, glycerate) could be produced in a growth and host metabolism independent manner using formaldehyde, formate or methanol as the sole C1 substrates. One can envision potential bioprocesses in which growth and maintenance is performed with a multi-carbon substrate, while the biocatalyst is used for C1 bioconversions. Bioprocesses of this nature, based on multi-enzyme cascades and two phase fermentations, have been the subject of recent reviews⁵⁷⁻⁵⁸.

While product synthesis from C1 substrates is a defining feature of FORCE pathways, they also have the potential to enable growth on non-native C1 substrates (e.g. synthetic methylotrophy) via the production of multi-carbon compounds naturally consumed by heterotrophs, such as glycolate, acetate, or glyceraldehyde. Genome scale modeling and flux balance analysis revealed that FORCE pathways are comparable or better than alternative approaches and guided the design. While the current pathway performance could not support the growth of a single strain of E. coli on C1 substrates, the orthogonal nature of the pathway allowed us to separate and evaluate the pathway limitations to growth on formate, formaldehyde, and methanol in separate strains of E. coli. The potential for FORCE pathways to enable methylotrophy allows for possible bioprocess implementations more similar to traditional fermentations based on C1 as a sole carbon source for both growth and product synthesis. Because the FORCE pathway is the branch point for fluxes toward product synthesis and growth, there is significant potential for facile control over flux partitioning (FIG. 1b), especially with recent developments in the area of dynamic metabolic control^{59,60}.

Further FORCE pathways development should enable more efficient designs for synthetic methylotrophy and diverse product synthesis, especially via pathway iteration. While the current demonstration enabled C1 utilization rates of 118 moles/OD/h when implemented at physiologically relevant concentrations of formaldehyde (FIG. 15b), we assess further improvements can be made by improving expression levels and kinetic parameters of HACL. The observation of formate as a byproduct throughout various implementations using formaldehyde or methanol is likely due to an imbalance between the rate of production of formyl-CoA and the rate of its utilization by HACL. We have also observed formyl-CoA hydrolysis¹⁶, which is probably exacerbated in vivo by endogenous thioesterases. Strategies to address this limitation include re-activating formate to formyl-CoA using a CoA-transferase, as done here using the CoA-transferase CaAbfT, and identification or engineering of an HACL enzyme with better characteristics, shown here via the identification of BsmHACL. Finally, host-strain modifications such as the deletion of endogenous aldehyde dehydrogenases and thioesterases were also explored for this purpose.

As HACL-catalyzed condensation and enzyme activity was only recently described, we expect that further genome mining, bioprospecting, enzyme engineering, and biochemical characterization will result in better performing variants, ultimately overcoming pathway bottlenecks. HACL variants with well-defined chain length and functional group specificities, in combination with compatible, specific termination enzymes, will allow for the production of specific products, analogous to what has been demonstrated with other platform pathways⁶¹⁻⁶³. These studies will also shed additional light on the role of formyl-CoA in metabolism, which is likely greater than the synthetic pathway described here. Recent reports have already contributed to the advancement of knowledge in this area^{17,64}, and further studies are likely to follow.

Methods:

Thermodynamic Calculations

Standard Gibbs free energies of reactions were found either from database sources (MetaCyc)⁶⁵ or by using the eQuilibrator biochemical thermodynamics calculator⁴¹. Min-max driving forces of pathways were calculated using a previously reported method⁴¹ implemented using MATLAB (Mathworks).

Flux Balance Analysis

Flux balance analysis was performed using the COBRA Toolbox⁶⁶ for MATLAB (Mathworks) with the Gurobi solver (Gurobi Optimization, LLC). Reactions enabling the various methylotrophy pathways (Table 3) were added or modified to the E. coli genome scale model iML1515⁵². The limits on the substrate exchange reactions were set to 10 mmol C/g DCW/hr for all C1 substrates.

Reagents

All chemicals were obtained from Fisher Scientific Co. and Sigma-Aldrich Co. unless otherwise specified. Primers were synthesized by Integrated DNA Technologies or by Eurofins Genomics. Restriction enzymes were obtained from New England Biolabs unless otherwise specified.

Genetic Methods

Genes non-native to E. coli were codon-optimized and synthesized by GeneArt (Thermo Fisher). E. coli genes were amplified from chromosomal DNA according to standard protocols⁶⁷. Plasmid-based gene expression was achieved by cloning the desired gene(s) into pCDFDuet-1 or pETDuet-1 (Novagen) digested with appropriate restriction enzymes and by using In-Fusion cloning technology (Clontech Laboratories, Inc.). Gene knockouts and genomic modifications were created using a CRISPR-Cas9-based system developed for E. coli. pCas and pTargetF were gifts from S. Yang (Addgene plasmids nos. 62225 and 62226, respectively). Plasmids and strains used in this study are listed in Table 2.

Evaluation of Core Pathway Module Using Purified Enzymes

Plasmids contain genes encoding RuHACL^{G390N}, LmACR, and OfFrc were cloned into pCDFduet-1, which were then transformed into E. coli BL21(DE3) for expression. Overnight cultures of the expression strains were grown in LB with 100 mg/L spectinomycin, which was used to inoculate 50 ml TB medium supplemented with 50 mg/L spectinomycin in a 250 ml baffled flask at 1%. The culture was grown at 30° C. and 250 r.p.m. in an orbital shaker until OD₆₀₀ reached 0.4-0.6, at which point expression was induced with 0.1 mM IPTG. Then, 24 h post inoculation, cells were harvested by centrifugation. The cell pellets were washed once with a cold 9 g/L NaCl solution and stored at −80° C. until needed.

The frozen cell pellets were resuspended in 10 mL of cold lysis buffer (50 mM NaPi pH 7.4, 300 mM NaCl, 20 mM imidazole), to which 250 U of Benzonase nuclease was added. The mixture was further treated by sonication on ice using a Cole-Parmer ultrasonic processor CPX130 (3 min with cycles of 5 seconds pulse on and 6 seconds pulse off, and amplitude set at 30%) and centrifuged at 7,500 g for 15 min at 4° C. The supernatant was applied to a chromatography column containing 5 ml Ni-NTA agarose resin (Qiagen, Inc.), which had been pre-equilibrated with the lysis buffer. The column was then washed first with 10 ml of the lysis buffer and then with 25 ml of wash buffer (50 mM NaPi pH 7.4, 300 mM NaCl, 70 mM imidazole). The His-tagged protein of interest was eluted with 20 ml elution buffer (50 mM NaPi pH 7.4, 300 mM NaCl, 250 mM imidazole). The eluate was collected and applied to a 10,000 molecular weight cut-off Amicon ultrafiltration centrifugal device (Millipore), and the concentrate (<300 μL) was washed twice with 4 ml of 50 mM KPi, 10% glycerol pH 7.4 for desalting. Protein concentrations were calculated using the Bradford Protein Assay (Bio-Rad) according to the manufacturers protocol. Purified protein was saved in 20 μl aliquots at −80° C. until needed.

To test the utilization of formaldehyde as the sole C1 substrate, the reaction was comprised of 50 mM KPi pH 7.4, 5 mM MgCl₂, 0.1 mM TPP, 1 mM NAD⁺, 2 mM CoASH, 1 μM RuHACL^{G390N}, 1 μM LmACR, and 100 mM FALD. To test the utilization of formate and formaldehyde as co-substrates, the reaction was comprised of 50 mM KPi pH 7.4, 5 mM MgCl₂, 0.1 mM TPP, 1 mM succinyl-CoA, 1 μM RuHACL^{G390N}, 2 μM OfFrc, 100 mM sodium formate, and 100 mM formaldehyde. To test the utilization of formate as the sole C1 substrate, the reaction was comprised of 50 mM KPi pH 7.4, 5 mM MgCl₂, 0.1 mM TPP, 1 mM NADH, 2 mM succinyl-CoA, 1 μM RuHACL^{G390N}, 2 μM OfFrc, 1 μM LmACR, and 100 mM sodium formate. As a control, a reaction comprised of 50 mM KPi pH 7.4, 5 mM MgCl₂, 0.1 mM TPP, 1 mM NADH, 1 mM NAD⁺, 2 mM succinyl-CoA, 2 mM CoASH, 2 μM BSA, 100 mM sodium formate, and 100 mM formaldehyde. The reaction volumes were 200 μL and the reactions were carried out at room temperature for 30 minutes on a rotisserie shaker.

Samples (200 μL) containing acyl-CoAs were first treated with 5 μL of 10 M NaOH to hydrolyze the thioesters and produce the carboxylic acid. Ammonium sulfate solution acidified with 1% sulfuric acid was then added to improve the efficiency of acid extraction. The resulting sample was extracted into 4 ml ethyl acetate by vigorous vortexing for 90 s. The organic phase was separated and evaporated to dryness under a stream of nitrogen. The residue was dissolved in 50 μl pyridine and 50 μl N,O-bis(trimethylsilyl)trifluoroacetamide, and incubated at 60° C. for 15 min. Derivatized samples were analyzed by GC-MS using an Agilent 5977B GC/MSD single quadrupole, Intuvo 9000 GC system, with integrated GERSTEL multifunctional autosampler sample preparation robot and an Agilent HP-5 ms capillary column (0.25 mm internal diameter, 0.25 μM film thickness, 30 m length). For the gas chromatography, 1 μL of the sample was injected with a 1:1 split ratio using helium as the carrier gas at a flowrate of 1.5 ml/min and the following temperature profile: initial 90° C. for 3 min; ramp at 15° C. per min to 170° C.; ramp at 20° C. per min to 300° C. and hold for 8 min. The injector and detector temperatures were 250 and 350° C., respectively. Data was acquired using Agilent MassHunter GC/MS Acquisition B.07.06.2704 and analyzed using Agilent MassHunter Workstation Software B.08.00.

To analyze the acyl-CoAs with LC-MS, the reaction was stopped by the adding 8 μL of formic acid to 200 μL reaction sample and desalted with 1 mL HyperSep C18 Cartridges (Thermo Scientific) that were primed twice with 200 μL methanol and equilibrated with 100 μL of 1 mM ammonium acetate pH 3.0. The columns were washed once with 200 μL of 1 mM ammonium acetate pH 3.0, and the acyl-CoAs were eluted in 200 μL methanol. LC-MS analysis was performed based on what has been previously described⁷. An Agilent 6540 Q-TOF LC-MS system was equipped with a Jet-stream electrospray ionization source set to the positive ionization mode and a 100 mm×4.6 mm Kinetex 2.6 μm Polar C18 100 Å column (Phenomenex). The LC conditions were: column oven set at 40° C., injection volume of 5 μL, and 50 mM ammonium formate and methanol as the mobile phases. Compound separation was achieved using the following gradient method at a flow rate of 400 μL/min: 0 min 0% methanol; 1 min 0% methanol; 3 min 2.5% methanol; 9 min 23% methanol; 14 min 80% methanol; 16 min 80% methanol; 17 min 0% methanol. The MS conditions were: capillary voltage 3.5 kV, nozzle voltage 500 V, fragmentor voltage 150 V, with nitrogen used for nebulizing (25 psig), drying (5 L/min, 225° C.), and sheath gas (10 L/min, 400° C.). A scan range of 100-1000 m/z was used. Data was acquired using Agilent MassHunter LC/MS data Acquisition B.05.01 and analyzed using MassHunter Qualitative Analysis B.05.00 (Agilent).

Cell-Free Metabolic Engineering for Pathway Validation

Enzyme expression and cell extract preparation was performed as described previously¹⁶. Cell-free reactions contained 50 mM KPi pH 7.4, 4 mM MgCl₂, 0.1 mM TPP, 2.5 mM CoASH, 5 mM NAD⁺, 50 mM formaldehyde, and 0.1 mM coenzyme B12. Individual cell extract loading was around 4.4 g/L protein (⅛ of the reaction volume), and the amount of protein added to each reaction was normalized with BL21(DE3) extract to ˜26 g/L protein (¾ of the reaction volume). The reactions were incubated at room temperature for the indicated time, at which point ¼ of the reaction volume of saturated ammonium sulfate solution acidified with 1% sulfuric acid was added to stop the reactions. Samples were centrifuged at 20817×g for 15 minutes and the supernatant analyzed by HPLC using a Shimadzu Prominence SIL 20 system (Shimadzu Scientific Instruments, Inc.) equipped with a refractive index detector and an HPX-87H organic acid column (Bio-Rad) with operating conditions to optimize peak separation (0.3 ml/min flowrate, 30 mM H₂SO₄ mobile phase, column temperature 42° C.). Data was acquired and analyzed using Shimadzu LabSolutions v5.96.

Resting Cell Bioconversions

Bioconversions using resting cells were performed as described previously¹⁶ with slight modification. The basal salts media used was M9 (6.78 g/L Na₂HPO₄, 3 g/L KH₂PO₄, 1 g/L NH₄Cl, 0.5 g/L NaCl, 2 mM MgSO₄, 100 μM CaCl₂, and 15 μM thiamine-HCl) additionally supplemented with the micronutrient solution of Neidhardt⁶⁸. An overnight LB culture of each strain was used to inoculate (1%) a 250 mL flask containing 50 mL of the above media further supplemented with 20 g/L glycerol, 10 g/L tryptone, 5 g/L yeast extract, and appropriate antibiotics (50 μg/mL carbenicillin, 50 μg/mL spectinomycin). The flask cultures were incubated at 30° C. and 250 rpm in an NBS 124 Benchtop Incubator Shaker (New Brunswick Scientific Co.). After 2.5 hours, gene expression was induced by addition of 0.1 mM isopropyl β-d-1-thiogalactopyranoside (IPTG) and 0.04 mM cumate (0.2 mM IPTG and 0.1 mM cumulate was used for the experiment with formaldehyde and formate).

The cells from the above cultures were harvested by centrifugation (5000×g, 22° C., 5 min), and washed twice with the above M9 media without any carbon source. The final cell pellet was resuspended in M9 with the appropriate carbon source (˜10 OD₆₀₀ with 10 mM formaldehyde or ˜5 OD₆₀₀ with 1 mM formaldehyde and 10 mM formate). 5 mL of the cell suspension was added to a 25 mL Erlenmeyer flask (Corning Inc.) and topped with a foam plug. Flasks were incubated at 30° C. and 200 rpm in an NBS 124 Benchtop Incubator Shaker (New Brunswick Scientific Co.). An additional 10 mM formaldehyde was added after 1.5 hours when formaldehyde was the sole carbon source. Samples were taken after 24 hours for HPLC analysis as described above. When 13C-labeled formaldehyde was used as the substrate, the samples were analyzed by GC-MS after extraction and derivatization as described above.

Fermentation Experiments

The growth media used was M9 (6.78 g/L Na₂HPO₄, 3 g/L KH₂PO₄, 1 g/L NH₄Cl, 0.5 g/L NaCl, 2 mM MgSO₄, 100 μM CaCl₂, and 15 μM thiamine-HCl) additionally supplemented with 500 mM methanol, 10 g/L tryptone, 5 g/L yeast extract and micronutrient solution of Neidhardt⁶⁸. An overnight LB culture of each strain was used to inoculate (1%) a 50 mL closed-cap conical tube (Genesee Scientific Co.) containing 5 mL of the above media further supplemented with appropriate antibiotics (50 μg/mL carbenicillin, 50 μg/mL spectinomycin). After approximately 3 hours, gene expression was induced by addition of 0.04 mM isopropyl β-d-1-thiogalactopyranoside (IPTG) and 0.04 mM cumate. Tubes were incubated at 30° C. and 200 rpm in an NBS 124 Benchtop Incubator Shaker (New Brunswick Scientific Co.). Samples (100 μL) were taken every 24, 48, 72 and 96 hours after inoculation for OD₆₀₀ measurement and HPLC analysis as described above. When 13C-methanol was used as the substrate, the samples were analyzed by GC-MS after extraction and derivatization as described above.

Two-Strain E. coli System for Growth on C1 Substrates

Two-strain experiments were conducted using strains cultured and induced as described previously using M9 medium¹⁶. The induced cells were resuspended to an initial concentration of 3*10⁹ CFU (colony forming unit)/mL (equivalent to OD₆₀₀ of ˜5) in M9 medium. 20 mL of the suspension was added into 25 mL flask containing 3 mg paraformaldehyde (equivalent to 5 mM), or 10 mL of the suspension was added into 25 mL flask with the addition of 500 mM methanol, or 1 mM formaldehyde and 10 mM sodium formate. A second E. coli strain, AC763, capable of consuming glycolate, was added to an initial concentration of 5*10⁶ CFU/mL (equivalent to OD₆₀₀ of ˜0.005). AC763 additionally harbored a chromosomal copy of constitutively expressed eGFP to assist in distinguishing the two strains. Prior to its addition to the culture, AC763 was pre-grown in 25 mL Erlenmeyer flasks (from a single colony inoculation) at 200 rpm and 30° C. for 24 hours in 5 mL of the above M9 minimal media supplemented with 5 g/L glycolate and 2 g/L tryptone. Cells were then centrifuged (5000×g, 22° C., 5 min), washed twice with the media supplemented with 5 g/L glycolate, and resuspended to an optical density of ˜0.05. Following 24 hours of incubation at 200 rpm and 30° C. (5 mL in 25 mL Erlenmeyer flasks), cells were centrifuged (5000×g, 22° C.), washed twice with media without any carbon source and an appropriate volume added to the two-strain system. The flasks containing both strains were further incubated at 200 rpm and 30° C. Samples were taken at various times for HPLC and cell growth analysis.

Colony forming units per mL of culture was utilized as a measurement of cell growth. Appropriate volumes of culture were diluted in the above described minimal media without any carbon source and 50 μL of various dilutions plated on minimal media plates containing 2.5 g/L glycolate. Following plate incubation at 37° C., colonies were counted manually, aided by visualization using a blue-light transilluminator (Vernier, Beaverton, OR) to illuminate the eGFP expressing strain AC763.

Data Availability Statement All data supporting the findings of this study are included herein as well as the following public databases: MetaCyc (metacyc.org/); eQuilibrator (equilibrator.weizmann.ac.il/); Uniprot (www.uniprot.org/). Uniprot accession numbers for enzymes involved in the study are given in Table 2. All Uniprot sequences are incorporated by reference in their entirety as listed in Table 2.

Code Availability Statement The scripts used to perform the analyses in the study are found at github.com/ahc7/FORCE_manuscript.

TABLE 1
Predicted carbon and electron biomass yields from various C1 substrates
by the implementation of different pathways enabling C1-trophy.

| | Biomass Carbon Yield (g DCW/mol C) | | | | | Biomass Electron Yield (g DCW/mol [2e−]) | | | | |
|---|---|---|---|---|---|---|---|---|---|---|
| | CO2 + H2 | | | | | CO2 + H2 | | | | |
| | (2:7) | Formate | Formald | Methanol | Methane | (2:7) | Formate | Formald | Methanol | Methane |
| FORCE - | 9.8 | 3.9 | 13.0 | 19.4 | 13.0 | 2.8 | 3.9 | 6.5 | 6.5 | 3.3 |
| Glycerald | | | | | | | | | | |
| Formolase | 9.7 | 3.8 | 12.8 | 19.1 | 12.8 | 2.8 | 3.8 | 6.4 | 6.4 | 3.2 |
| RuMP | 9.7 | 3.8 | 12.8 | 19.1 | 12.8 | 2.8 | 3.8 | 6.4 | 6.4 | 3.2 |
| WL* | 9.3 | 3.7 | 12.4 | 18.4 | 12.4 | 2.7 | 3.7 | 6.2 | 6.1 | 3.1 |
| RTCA | 9.2 | 3.6 | 12.2 | 18.2 | 12.2 | 2.6 | 3.6 | 6.1 | 6.1 | 3.0 |
| FORCE - | 9.1 | 3.6 | 12.1 | 18.0 | 12.1 | 2.6 | 3.6 | 6.0 | 6.0 | 3.0 |
| Ac | | | | | | | | | | |
| SACA | 9.1 | 3.6 | 12.1 | 18.0 | 12.1 | 2.6 | 3.6 | 6.0 | 6.0 | 3.0 |
| Reductive | 8.9 | 3.5 | 11.7 | 17.5 | 11.7 | 2.5 | 3.5 | 5.9 | 5.8 | 2.9 |
| Glycine | | | | | | | | | | |
| Serine | 8.5 | 3.4 | 11.2 | 16.8 | 11.2 | 2.4 | 3.4 | 5.6 | 5.6 | 2.8 |
| FORCE - | 8.4 | 3.3 | 11.1 | 16.5 | 11.1 | 2.4 | 3.3 | 5.5 | 5.5 | 2.8 |
| Glycolate | | | | | | | | | | |
| DC4HB | 7.8 | 3.1 | 10.3 | 15.4 | 10.3 | 2.2 | 3.1 | 5.1 | 5.1 | 2.6 |
| CBB | 7.5 | 3.0 | 9.9 | 14.8 | 9.9 | 2.1 | 3.0 | 5.0 | 4.9 | 2.5 |
| CETCH | 6.7 | 2.7 | 8.9 | 13.3 | 8.9 | 1.9 | 2.7 | 4.4 | 4.4 | 2.2 |

*Ferredoxin-dependent reactions were replaced with NAD+/NADH for integration with the E. coli genome scale model and to avoid applying a potentially pathway-independent penalty associated with the ability of E. coli to regenerate the ferredoxin electron acceptor.

TABLE 2
Host strains and plasmids used in this study. Uniprot accession numbers
for heterologous enzymes used in this work are given in parenthesis.

| Host Strains/ | | |
|---|---|---|
| Plasmids | Description/Genotype/Usage | Source |
| BL21(DE3) | E. coli B F⁻ ompT gal dcm lon hsdS_{B}(r_{B}⁻m_{B}⁻) [malB⁺]_{K-12}(λ^{S}) | Studier et al. ⁶⁹ |
| | λ(DE3) | |
| | Host for protein expression for in vitro studies | |
| MG1655 | E. coli K-12 F- I- ilvG- rfb-50 rph-1 | Blattner et al.⁷⁰ |
| AC440 | MG1655 λ(DE3) ΔfrmA ΔfdhF ΔfdnG ΔfdoG ΔglcD::FRT | Chou et al.¹⁶ |
| | Engineered host for resting cell studies | |
| AC877 | AC440 ΔaldA ΔaldB ΔpatD ΔpuuC | This study |
| | Engineered host for resting cell studies | |
| AC878 | AC440 ΔyciA ΔtesA ΔtesB ΔybgC ΔydiI ΔfadM | This study |
| | Engineered host for methanol utilization | |
| AC763 | MG1655 λ(DE3) ΔfrmA ΔfdhF ΔfdnG ΔfdoG ΔtesB::P^{M193}-eGFP | This study |
| | C2-utilizing (sensor) strain for two-strain pathway evaluation. | |
| | The tesB open reading frame was replaced with eGFP | |
| | controlled by constitutive promoter M193⁷¹ | |
| pCDFDuet-1 | CloDF13, lacI, Sm^{R} | Novagen |
| | | (Darmstadt, |
| | | Germany) |
| pCDFDuet-1-P1- | pCDFDuet-1 with codon optimized 6xHis-tagged | Chou et al.¹⁶ |
| ntH6-RuHACL^{G390N} | Rhodospirillales bacterium URHD0017 HACL (Uniprot: | |
| | A0A1H8YFL8) with a G390N mutation under control of the T7lac | |
| | promoter and lacI | |
| pCDFDuet-1-P1- | pCDFDuet-1 with codon optimized 6xHis-tagged | This study |
| ntH6- | Rhodospirillales bacterium URHD0017 HACL with a G390N | |
| RuHACL^{G390N}-P2- | mutation in the P1 cloning site and codon optimized Bacillus | |
| BmMDH2^{MGA3} | methanolicus MGA3 MDH2 (Uniprot: I3E2P9) in the P2 cloning | |
| | site both under control of the T7lac promoter and lacI | |
| pCDFDuet-1-P1- | pCDFDuet-1 with codon optimized Bacillus methanolicus MGA3 | This study |
| P2-BmMDH2^{MGA3} | MDH2 in the P2 cloning site both under control of the T7lac | |
| | promoter and lacI | |
| pCDFDuet-1-P1- | pCDFDuet-1 with codon optimized 6xHis-tagged HACL isolated | This study |
| ntH6-BsmHACL | from beach sand metagenome (UniProt: A0A3C0TX30) under | |
| | control of the T7lac promoter and lacI | |
| pCDFDuet-1-P1- | pCDFDuet-1 with codon optimized 6xHis-tagged HACL isolated | This study |
| ntH6-BsmHACL- | from beach sand metagenome in the P1 cloning site and codon | |
| P2-BmMDH2^{MGA3} | optimized Bacillus methanolicus MGA3 MDH2 in the P2 cloning | |
| | site both under control of the T7lac promoter and lacI | |
| pCDFDuet-1-P1- | pCDFDuet-1 with codon optimized 6xHis-tagged HACL isolated | This study |
| ntH6-BsmHACL- | from beach sand metagenome in the P1 cloning site and a | |
| P2-BmMDH2^{MGA3}- | synthetic operon of codon optimized Bacillus methanolicus | |
| CaAbfT | MGA3 MDH2 and codon optimized Clostridium aminobutyricum | |
| | abfT (UniProt: Q9RM86) in the P2 cloning site under control of | |
| | the T7lac promoter and lacI | |
| pCDFDuet-1-P1- | pCDFDuet-1 with codon optimized 6xHis-tagged Lysteria | Chou et al.¹⁶ |
| ntH6-LmACR | monocytogenes acr (Uniprot: Q8Y7U1) under control of the | |
| | T7lac promoter and lacI | |
| pCDFDuet-1-P1- | pCDFDuet-1 with codon optimized 6xHis-tagged Oxalobacter | This study |
| ntH6-OfFrc | formigenes frc (Uniprot: O06644) under control of the T7lac | |
| | promoter and lacI | |
| pETDuet-1 | pBR322-derived ColE1 origin, lacI, Amp^{R} | Novagen |
| | | (Darmstadt, |
| | | Germany) |
| pETDuet-1-P1- | pETDuet-1 with Escherichia coli fucO in the P1 cloning site | This study |
| EcFucO | under control of the T7lac promoter and lacI | |
| pETDuet-1-P^{CT5}- | pETDuet-1 with codon optimized Lysteria monocytogenes acr | Chou et al.¹⁶ |
| LmACR | expressed under control of the cumate inducible CT5 promoter | |
| | and cymR | |
| pETDuet-1-P^{CT5}- | pETDuet-1 with codon optimized Lysteria monocytogenes acr | Chou et al.¹⁶ |
| LmACR-EcAldA | and Escherichia coli aldA in a synthetic operon under control of | |
| | the cumate inducible CT5 promoter and cymR | |
| pETDuet-1-P^{CT5}- | pETDuet-1 with codon optimized Lysteria monocytogenes acr | This study |
| LmACR-EcFucO | and Escherichia coli fucO in a synthetic operon under control of | |
| | the cumate inducible CT5 promoter and cymR | |
| pETDuet-1-P^{CT5}- | pETDuet-1 with codon optimized Clostridium aminobutyricum | This study |
| CaAbfT | abfT under control of the cumate inducible CT5 promoter and | |
| | cymR | |
| pRSFDuet-1 | pRSF1030-derived RSF origin, lacI, KanR | Novagen |
| | | (Darmstadt, |
| | | Germany) |
| pRSFDuet-1-P1- | pRSFDuet-1 with codon optimized 6xHis-tagged Klebsiella | This study |
| ntH6-KoPddABC- | oxytoca pddA (Uniprot: Q59470) in a synthetic operon with | |
| P2-KoDdrAB- | codon optimized Klebsiella oxytoca pddB (Uniprot: Q59471) and | |
| EcYciK-EcBtuR | pddC (Uniprot: Q59472) in the P1 cloning site and codon | |
| | optimized Klebsiella oxytoca ddrA (Uniprot: O68195) and ddrB | |
| | (Uniprot: O68196) in a synthetic operon with Escherichia coli | |
| | yciK and btuR in the P2 cloning site both operons under control | |
| | of the T7lac promoter and lacI | |

TABLE 3
Modifications made to iML1515 to implement methylotrophic pathways. ‘L’
refers to the lower limit of reaction flux, ‘U’ refers to the upper
limit of reaction flux, ‘B’ refers to both upper and lower limits of reaction flux.

| | | | |
|---|---|---|---|
| Reaction name | Reaction | Modification | Description |

Global modifications

| | | | |
|---|---|---|---|
| FORtppi | for_c <=> for_p | L = −1000 | Allow passive formate |
| | | | import⁷² |
| EX_glc_{——}D_e | glc_{——}D_e <=> | L = 0 | Remove glucose input |
| FDH | h_c + nadh_c + | Add | NAD-dependent formate |
| | co2_c <=> nad_c + | | dehydrogenase |
| | for_c | | |
| formylKinase | atp_c + for_c <=> | Add | Formate activation (1 |
| | adp_c + forp_c | | ATP) |
| formylTransferase | coa_c + forp_c <=> | Add | Formate activation (1 |
| | pi_c + forcoa_c | | ATP) |
| acylAldRed | nad_c + fald_c + | Add | Conversion of |
| | coa_c <=> h_c + | | formaldehyde and |
| | forcoa_c + nadh_c | | formyl-CoA |
| MeOHDH | nad_c + MeOH_c <=> | Add | Methanol dehydrogenase |
| | h_c + fald_c + | | |
| | nadh_c | | |
| hydrogenase | nad_c + h2_c <=> | Add | NAD-dependent |
| | h_c + nadh_c | | hydrogenase |

FORCE-glycolate model

| | | | |
|---|---|---|---|
| HACL | fald_c + forcoa_c <=> | Add | HACL-catalyzed reaction |
| | glyclcoa_c | | |
| glycltcoaTes | h2o_c + glyclcoa_c <=> | Add | Hydrolysis of glycolyl- |
| | glyclt_c + coa_c | | CoA to glycolate |

FORCE-acetate model

| | | | |
|---|---|---|---|
| HACL | fald_c + forcoa_c <=> | Add | HACL-catalyzed reaction |
| | glyclcoa_c | | |
| glycltcoaTes | h2o_c + glyclcoa_c <=> | Add | Hydrolysis of glycolyl- |
| | glyclt_c + coa_c | | CoA to glycolate |
| ACR | h_c + nadh_c + | Add | Reduction of glycolyl- |
| | glyclcoa_c <=> nad_c + | | CoA to glycolaldehyde |
| | gcald_c + coa_c | | |
| DOR | h_c + gcald_c + | Add | Conversion of |
| | nadh_c <=> nad_c + | | glycolaldehyde and |
| | ethgly_c | | ethylene glycol |
| DDR | ethgly_c −> h2o_c + | Add | Dehydration of ethylene |
| | acald_c | | glycol |

FORCE-glyceraldehyde model

| | | | |
|---|---|---|---|
| HACL | fald_c + forcoa_c <=> | Add | HACL-catalyzed reaction |
| | glyclcoa_c | | |
| ACR | h_c + nadh_c + | Add | Reduction of glycolyl- |
| | glyclcoa_c <=> nad_c + | | CoA to glycolaldehyde |
| | gcald_c + coa_c | | |
| HACLC3 | forcoa_c + gcald_c <=> | Add | HACL iteration to C3 |
| | glycercoa_c | | |
| glycercoaTes | h2o_c + glycercoa_c <=> | Add | Hydrolysis of glyceryl- |
| | glyc_{——}R_c + coa_c | | CoA |
| glycercoaRed | h_c + nadh_c + | Add | Reduction of glyceryl- |
| | glycercoa_c <=> | | CoA |
| | glyald_c + nad_c + | | |
| | coa_c | | |

RUMP model

| | | | |
|---|---|---|---|
| HPS | fald_c + ru5p_{——}D_c <=> | Add | 3-hexulose-6-phosphate |
| | h6p_c | | synthase |
| PHI | h6p_c <=> f6p_c | Add | 6-phospho-3- |
| | | | hexuloisomerase |

Serine Cycle model

| | | | |
|---|---|---|---|
| THFlig | fald_c + thf_c <=> | Add | Ligation of formaldehyde |
| | mlthf_c + h2o_c | | and tetrahydrofolate |
| SGA | ser_{——}L_c + glx_c <=> | Add | Serine-glyoxylate |
| | hpyr_c + gly_c | | aminotransferase |
| MTK | mal_{——}L_c + atp_c + | Add | Malate |
| | coa_c −> adp_c + | | thiokinase |
| | pi_c + malylcoa_c | | |
| MCL | malylcoa_c <=> | Add | Malyl-CoA lyase |
| | accoa_c + glx_c | | |

Formolase model

| | | | |
|---|---|---|---|
| FLS | 3 fald_c <=> dha_c | Add | Formolase reaction |

SACA pathway model

| | | | |
|---|---|---|---|
| GALS | 2 fald_c <=>gcald_c | Add | Glycolaldehyde synthase |
| ACPS | pi_c + gcald_c <=> | Add | Acetyl-phosphate |
| | actp_c + h2o_c | | synthase |

Reductive Glycine model

| | | | |
|---|---|---|---|
| GLYCL | nad_c + thf_c + | L = −1000 | Reversal of glycine |
| | gly_c −> nh4_c + | | cleavage |
| | mlthf_c + nadh_c + | | |
| | co2_c | | |

Formate utilization models

| | | | |
|---|---|---|---|
| EX_for_e | for_e −> | L = −10 | Allow formate input |
| FDH | h_c + nadh_c + | U = 0 | Prevent reutilization of |
| | co2_c <=> nad_c + | | CO₂ by direct reduction |
| | for_c | | |

Formaldehyde utilization models

| | | | |
|---|---|---|---|
| EX_fald_e | fald_e −> | L = −10 | Allow formaldehyde input |
| FDH | h_c + nadh_c + | U = 0 | Prevent reutilization of |
| | co2_c <=> nad_c + | | CO₂ by direct reduction |
| | for_c | | |
| formylKinase | atp_c + for_c <=> | B = 0 | Prevent reutilization of |
| | adp_c + forp_c | | oxidized carbon |
| formylTransferase | coa_c + forp_c <=> | B = 0 | Prevent reutilization of |
| | pi_c + forcoa_c | | oxidized carbon |

Methanol utilization models

| | | | |
|---|---|---|---|
| EX_MeOH_e | MeOH_e <=> | Add, L = −10 | Allow methanol input |
| MeOHIn | MeOH_e <=> MeOH_c | Add | Allow methanol import to |
| | | | cytoplasm (simplified) |
| FDH | h_c + nadh_c + co2_c <=> | U = 0 | Prevent reutilization of |
| | nad_c + for_c | | CO₂ by direct reduction |
| formylKinase | atp_c + for_c <=> | B = 0 | Prevent reutilization of |
| | adp_c + forp_c | | oxidized carbon |
| formylTransferase | coa_c + forp_c <=> | B = 0 | Prevent reutilization of |
| | pi_c + forcoa_c | | oxidized carbon |

TABLE 4
Comparison of the properties of selected C1-utlization pathways with potential
to enable methylotrophy (from methanol) to a representative C2 (acetyl-CoA) and
C3 (pyruvate) metabolite. The pathways from this work used for the calculations
were: to acetyl-CoA via acetaldehyde produced from diol dehydratase and to pyruvate
via glycerate. Positive values indicate net production, while negative values indicate
net consumption. Calculations assume the hydrolysis of ATP to AMP to be two equivalents
of ATP to ADP hydrolysis. Carbon yield for methylotrophic pathways is based on moles
of methanol in the C2 or C3 metabolite and thus is greater than 100% in cases where
CO₂ is also assimilated (i.e. serine pathway).

| | | | | | | # |
|---|---|---|---|---|---|---|
| | | Net redox | Net ATP | Oxygen | Carbon yield | Reactions |
| Pathway | Origin | (C2/C3) | (C2/C3) | sensitivity | (C2/C3) | (C2/C3) |
| This work | Engineered | +2/+4 | 0/0 | Partial | 100%/100% | 7/9 |
| RuMP | Bacterial | +5/+4 | +1/0 | None | 67%/100% | 17/10 |
| Serine | Bacterial | −1/+1 | −2/−2 | None | 200%/150% | 12/15 |
| XuMP | Eukaryal | +5/+4 | −1/−1 | None | 67%/100% | 16/15 |
| Formolase⁶ | Engineered | +5/+4 | +1/+1 | None | 67%/100% | 10/9 |
| MCC⁵ + | Engineered | +2/+7 | 0/0 | None | 100%/75% | 10/19 |
| Glyoxylate | | | | | | |
| bypass | | | | | | |
| SACA⁸ + | Engineered | +2/+7 | 0/0 | None | 100%/75% | 4/13 |
| Glyoxylate | | | | | | |
| bypass | | | | | | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A non-natural microbial system capable of utilizing one-carbon (C1) substrates for growth and product synthesis, comprising:
a first set of nucleic acids encoding enzymes to convert the single carbon substrate to formyl-CoA and formaldehyde; and
a second set of nucleic acids encoding enzymes to convert formyl-CoA and formaldehyde to native multi-carbon substrates or metabolites that enable growth.

2. The system of claim 1, wherein the system comprises a one carbon substrate, optionally, wherein the C1 substrate comprises methane.

3. The system of claim 2, wherein the first set of metabolic enzymes comprises a methane monooxygenase that can convert the methane to methanol, a methanol dehydrogenase that can convert the methanol to formaldehyde, and an acyl-CoA reductase that can convert the formaldehyde to formyl-CoA.

4. The system of claim 1, wherein the C1 substrate comprises carbon dioxide.

5. The system of claim 4, wherein the first set of metabolic enzymes comprises a formate dehydrogenase that can convert the carbon dioxide to formate.

6. The system of claim 5, wherein the first set of metabolic enzymes further comprises an enzyme that can convert the formate to formyl-CoA.

7. The system of claim 5, wherein the first set of metabolic enzymes further comprises a formaldehyde dehydrogenase that can convert formate to formaldehyde, and an acyl-CoA reductase that can convert the formaldehyde to formyl-CoA.

8. The system of claim 5, wherein the first set of metabolic enzymes further comprises a formate kinase that can convert the formate to formyl-phosphate and a phosphotransacylase that can convert the formyl-phosphate to formyl-CoA.

9. The system of claim 1, wherein the C1 substrate comprises formate.

10. The system of claim 9, wherein the first set of metabolic enzymes comprises an enzyme that can convert the formate to formyl-CoA.

11. The system of claim 9, wherein the first set of metabolic enzymes comprises a formaldehyde dehydrogenase that can convert formate to formaldehyde, and an acyl-CoA reductase that can convert the formaldehyde to formyl-CoA.

12. The system of claim 9, wherein the first set of metabolic enzymes comprises a formate kinase that can convert the formate to formyl-phosphate and a phosphotransacylase that can convert the formyl-phosphate to formyl-CoA.

13. The system of claim 1, wherein the C1 substrate comprises carbon monoxide.

14. The system of claim 13, wherein the first set of metabolic enzymes comprises a carbon monoxide dehydrogenase that can convert the carbon monoxide to carbon dioxide, and a formate dehydrogenase that can convert the carbon dioxide to formate.

15. The system of claim 14, wherein the first set of metabolic enzymes further comprises an enzyme that can convert the formate to formyl-CoA.

16. (canceled)

17. (canceled)

18. The system of claim 1, wherein the C1 substrate comprises methanol.

19. (canceled)

20. The system of claim 1, wherein the C1 substrate comprises formaldehyde.

21. (canceled)

22. The system of claim 1, wherein the second set of metabolic enzymes comprises 2-hydroxyacyl-CoA lyase (HACL).

23. The system of claim 22, wherein the second set of metabolic enzymes further comprises an acyl-CoA reductase.

24. The system of claim 23, wherein the second set of metabolic enzymes further comprises a 1.2-diol oxidoreductase.

25-73. (canceled)
